(19) United States
(12) Patent Application Publication
(10) Pub. No.: US 2021/0370299 A1
(43) Pub. Date: Dec. 2, 2021

(54) THERMAL ASSEMBLIES FOR NUCLEIC ACID PREPARATION

(71) Applicant: ArcherDX, LLC, San Francisco, CA (US)

(72) Inventors: Joshua Stahl, Boulder, CO (US); Jason Myers, Boulder, CO (US)

(73) Assignee: ArcherDX, LLC, San Francisco, CA (US)

(21) Appl. No.: 16/336,345

(22) PCT Filed: Sep. 22, 2017
(86) PCT No.: PCT/US2017/053097

§ 371 Date: Mar. 25, 2019

Related U.S. Application Data
(63) Continuation of application No. PCT/US2017/051924, filed on Sep. 15, 2017.
Application No. PCT/US2017/051924 is a continuation of application No. PCT/US2017/051927, filed on Sep. 15, 2017.
(60) Provisional application No. 62/398,841, filed on Sep. 23, 2016.
(60) Provisional application No. 62/399,152, filed on Sep. 23, 2016.
(60) Provisional application No. 62/399,157, filed on Sep. 23, 2016.
(60) Provisional application No. 62/399,184, filed on Sep. 23, 2016.
(60) Provisional application No. 62/399,195, filed on Sep. 23, 2016.
(60) Provisional application No. 62/399,205, filed on Sep. 23, 2016.
(60) Provisional application No. 62/399,211, filed on Sep. 23, 2016.
(60) Provisional application No. 62/399,219, filed on Sep. 23, 2016.

(51) Int. Cl. B01L 3/00; B01L 7/00; B01L 7/04; H05K 1/02; H05K 7/20; H05K 3/30

(52) U.S. Cl. CPC B01L 3/502761; B01L 2300/1883; B01L 7/04; H05K 1/0204; H05K 7/2039; H05K 3/306; B01L 2300/1822; B01L 2300/12; B01L 2300/02; B01L 2200/12; B01L 2200/0668; B01L 2300/0816; B01L 2300/0887; B01L 2300/041; B01L 2200/027; B01L 7/52

(57) ABSTRACT

Provided herein are apparatus for independently manipulating the temperature of a plurality of reaction vessels, e.g., for automated processing of nucleic acids present in the vessels. Printed circuit boards (PCBs) comprising a mount area arranged to have mounted thereon a through-hole thermoelectric device (TED) to facilitate independent temperature control of reaction vessels are also provided, as well as methods relating to the same.

### RELATED APPLICATIONS

This application claims priority under 35 U.S.C. § 119(e) to U.S. Provisional Patent Application Nos. 62/398,841, 62/399,152, 62/399,157, 62/399,184, 62/399,195, 62/399,205, 62/399,211, and 62/399,219, each of which was filed on Sep. 23, 2016, and claims priority under 35 U.S.C. §§ 120 and 365(c) to PCT International Application No. PCT/US2017/051924, which was filed on Sep. 15, 2017, and which claims priority under 35 U.S.C. § 119(e) to U.S. Provisional Patent Application No. 62/395,339, which was filed on Sep. 15, 2016, and to PCT International Application No. PCT/US2017/051927, which was filed on Sep. 15, 2017, and which claims priority under 35 U.S.C. § 119(e) to U.S. Provisional Patent Application No. 62/395,347, which was filed on Sep. 15, 2016, the entire contents of each of which applications are hereby incorporated by reference.

### TECHNICAL FIELD

The present invention generally relates to systems and related methods for automated processing of molecules (e.g., nucleic acids).

### BACKGROUND

Numerous approaches for processing nucleic acids have been developed. Such methods often included multiple enzymatic, purification, and preparative steps that make them laborious and prone to error, including errors associated with contamination, systematic user errors, and process biases. As a result, it is often difficult to execute such processes reliably and reproducibly, particularly when the processes are being conducted commercially, e.g., in a multiplex or high-throughput context.

### SUMMARY

The present invention generally relates to systems, apparatus and related methods for processing nucleic acids. In some embodiments, apparatus are provided for independently manipulating the temperature of a plurality of reaction vessels, e.g., for automated processing of nucleic acids present in the vessels. Certain aspects of the invention relate to systems with independently manipulatable thermoelectric devices (e.g., TECs) for heating or cooling multiple reaction vessels for processing nucleic acids (e.g., for library preparation, amplification, sequencing and other similar processing techniques). In some embodiments, systems provided herein comprise cartridges including cassettes and/or microfluidic channels that facilitate automated processing of nucleic acids, including automated nucleic acid library preparations. In some embodiments, systems and related methods are provided for automated processing of nucleic acids to produce material for next generation sequencing and/or other downstream analytical techniques.

In some embodiments, apparatus are provided for independently manipulating the temperature of a plurality of reaction vessels that include a base assembly comprising a plurality of receptacles (e.g., 6, 12, 24, 36, 64, 96, 384 thermoelectric devices). In some embodiments, each receptacle is configured to have disposed therein a reaction vessel. In some embodiments, the base assembly further comprises a plurality of thermoelectric devices (e.g., 6, 12, 24, 36, 64, 96, 384 thermoelectric devices) configured to heat or cool each receptacle/reaction vessel. In some embodiments, each receptacle comprises a thermal jacket in thermal communication with at least one of the plurality of thermoelectric devices. In certain embodiments, a thermal jacket has a thermal transfer surface (e.g., a first thermal transfer surface) configured to surround at least a portion of a reaction vessel to facilitate thermal exchange between the reaction vessel and the jacket. In some embodiments, a first thermal transfer surface contacts a reaction vessel. In some embodiments, the first thermal transfer surface designed to control thermal contact conductance between the jacket and vessel (e.g., by having a smoothed surface). In some embodiments, the first thermal transfer surface is configured to surround a portion of the outer circumference of the reaction vessel.

In some embodiments, a thermal jacket has a thermal transfer surface (e.g., a second thermal transfer surface) that interfaces with the at least one thermoelectric device to facilitate heat exchange between the thermal jacket and the at least one thermoelectric device. In some embodiments, a thermal jacket has a thermal transfer surface (e.g., a second thermal transfer surface) in contact with a thermal pad that interfaces with the at least one thermoelectric device to facilitate heat exchange between the thermal jacket and the at least one thermoelectric device. In some embodiments, a “gap filler pad” is provided. In some embodiments, the gap filler pad is conductive and compressible, and used to accommodate variation in clamping pressure and variation in component thicknesses. In some embodiments, a thermally resistive pad is provided to isolate each TEC. In some embodiments, the thermal transfer surface and/or thermal pad is configured to control thermal contact conductance between the jacket and thermal pad (e.g., by having a smoothed surface). In some embodiments, the thermal pad is composed of silver, bronze, brass, aluminum, copper, steel, or other thermally conductive material. In some embodiments, a Lairdtech Tgard™ thermal pad (or suitable alternative) may be used. In some embodiments, one or more of various thermally conductive thermal pastes may also be used. Examples of thermally insulating materials are provided at: http://www.lairdtech.com/product-categories/thermal-management/thermal-materials/electrically-insulating. In some embodiments, the thermal pad has a thermal resistance (Modified ASTM D5470) in a range of 3° C.-cm²/watt to 6° C.-cm²/watt. In some embodiments, the thermoelectric device is referred to as a “thermoelectric coolers”. In some embodiments, the thermoelectric device is a solid-state heat pump that operates according to a Peltier effect, in which heating or cooling occurs when electric current passes through two conductors comprising dissimilar materials.

In some embodiments, the apparatus further comprise a cassette assembly comprising a plurality of reaction vessels. In some embodiments, the cassette assembly is configured and arranged to interface with the base assembly such that each of the plurality of receptacles of the base assembly has disposed therein a corresponding reaction vessel of the cassette assembly. In some embodiments, the apparatus further comprise a plurality of cassette assemblies, each cassette assembly comprising a plurality of reaction vessels. In some embodiments, each cassette assembly is configured and arranged to interface with the base assembly such that each of the plurality of receptacles of the base assembly has disposed therein a corresponding reaction vessel of the cassette assembly. In some embodiments, the cassette assemblies are disposed in a cartridge. In some embodiments, the cartridge comprises a support structure comprising a channel apparatus having a plurality of fluidic conduits. In some embodiments, one or more fluidic conduits is in fluidic communication with a reaction vessel. In some embodiments, the cartridge comprises a support structure comprising a channel apparatus having a plurality of fluidic conduits. In some embodiments, one or more fluidic conduits comprises a fluid outlet orifice that fluidically interfaces with an inlet port at the base of a reaction vessel.

In some embodiments, the support structure has a plurality of openings aligned with one or more reaction vessels. In some embodiments, each opening is configured to permit passage of a thermal jacket through the support structure to access and surround the reaction vessel. In some embodiments, the support structure comprises a plurality of elongate members, in which each elongate member extends from an outer edge of an opening to an inner position of the opening. In some embodiments, a fluidic conduit of the channel apparatus extends through the elongate member from the outer edge to the inner position ending at a fluid outlet orifice at the inner position. In some embodiments, the thermal jacket has a keyway that aligns with the elongate member to permits passage of the jacket through the opening to access and surround the jacket.

In some embodiments, the apparatus further comprises a thermal cover assembly. In some embodiments, the thermal cover assembly is configured to provide a heat source above each reaction vessel to prevent or minimize evaporation of a reaction solvent present in the reaction vessel. In some embodiments, the reaction solvent is water. In some embodiments, the thermal cover assembly has at least two independently thermally controlled zones.

According to certain aspects of the invention, apparatus are provided for managing temperature of one or more reaction vessels. In some embodiments, In some embodiments, the apparatus comprise a plurality of receptacles. In some embodiments, one or more receptacles is configured to have disposed therein a reaction vessel. In some embodiments, one or more receptacles comprises a thermal jacket in thermal communication with a thermoelectric device. In some embodiments, the thermal jacket has a thermal transfer surface (e.g., a first thermal transfer surface) configured to surround at least a portion of a reaction vessel to facilitate thermal exchange between the reaction vessel and the jacket. In some embodiments, a thermal cover assembly is configured to provide a heat source above each reaction vessel to prevent or minimize evaporation of a reaction solvent present in the reaction vessel. In some embodiments, the thermal cover assembly has at least two (e.g., 2, 3, 4, 5, 6, 7, 8, 9, 10 or more) independently thermally controlled zones. In some embodiments, the thermal cover assembly has two independently thermally controlled zones. In some embodiments, one or more thermally controlled zone aligns with one or more reaction vessels of the plurality of reaction vessels.

In some embodiments, the thermal cover assembly is configured with a plurality of openings to permit the passage of light. In some embodiments, each opening is optically aligned above a corresponding reaction vessel to permit light to pass into and out from the reaction vessel. In some embodiments, the openings permit optical measurements from each reaction vessel.

According to some aspects of the invention, methods of manufacturing a circuit having a through-hole thermoelectric device (TED) mounted to a printed circuit board (PCB) are provided. In some embodiments, the through-hole TEC is packaged with a through-hole mount packaging. In some embodiments, the through-hole TED comprises a first side having a first surface area. In some embodiments, the methods comprises mounting the through-hole TED to the PCB in a mount area of the PCB arranged for mounting of the through-hole TED to the PCB. In some embodiments, the mount area comprises first holes corresponding to through-hole leads of the through-hole TED and a second hole having a size matching the first surface area of the first side of the through-hole TED. In some embodiments, mounting the through-hole TED to the PCB comprises attaching the through-hole leads of the through-hole TED to the PCB via the first holes. In some embodiments, mounting the through-hole TED to the PCB further comprises arranging the through-hole TED on the PCB such that the first side of the through-hole TED is aligned with the second hole of the mounting area. In some embodiments, the through-hole TED is a thermoelectric cooler (TEC) packaged with the through-hole mount packaging.

In some embodiments, the through-hole TED is arranged to create a temperature differential between the first side of the through-hole TED and a second side of the through-hole TED based on a power applied to the through-hole TED. In some embodiments, the through-hole TED is arranged to create a thermal gradient in the through-hole TED via a Peltier effect. In some embodiments, the through-hole TED is operable to adjust a temperature of the first side of the through-hole TED in response to application of power to the through-hole leads.

In some embodiments, the manufacturing methods further comprise arranging the through-hole TED on the PCB such that the first side is aligned with the second hole comprises arranging the through-hole TED such that the first side is disposed in the second hole. In some embodiments, the methods further comprise arranging the through-hole TED on the PCB such that the first side is aligned with the second hole comprises arranging the through-hole TED such that a plane of the first side of the through-hole TED is substantially parallel to a plane of the second hole.

In some embodiments, the PCB comprises a plurality of the mount area, each mount area of the plurality comprising the first holes and the second hole. In some embodiments, the manufacturing methods further comprise repeating the mounting for a plurality of through-hole TEDs to mount the plurality of through-hole TEDs in the plurality of mount areas. In some embodiments, the mounting step is performed by circuit manufacture equipment configured to perform the mounting. In some embodiments, the holes each comprise an electrically conductive material individually electrically connecting the first holes to conductive traces of the PCB. In some embodiments, the step of attaching the through-hole leads of the through-hole TED to the PCB via the first holes comprises soldering a lead of the through-hole leads to the electrically conductive material of a hole of the first holes.

According to some aspects of the invention, apparatus are provided that comprise a printed circuit board (PCB) comprising a mount area arranged to have mounted thereon a through-hole thermoelectric device (TED). In some embodiments, the through-hole TED is packaged with a through-hole mount packaging. In some embodiments, the mount area comprises first holes corresponding to through-hole leads of the through-hole TED and a second hole having a size matching a first surface area of a first side of the through-hole TED. In some embodiments, the apparatus further comprises the through-hole TED, in which the through-hole TED is a thermoelectric cooler (TEC) packaged with the through-hole mount packaging.

In some embodiments, the through-hole TED is arranged to create a temperature differential between the first side of the through-hole TED and a second side of the through-hole TED based on a power applied to the through-hole TED. In some embodiments, the through-hole TED is arranged to create a thermal gradient in the through-hole TED via a Peltier effect. In some embodiments, the through-hole TED is operable to adjust a temperature of the first side of the through-hole TED in response to application of power to the through-hole leads.

In some embodiments, the apparatus comprises a through-hole TED mounted in the mount area of the PCB. In certain embodiments, the through-hole TED is mounted to the PCB with through-hole pins that are attached to the first holes of the PCB. In some embodiments, the first side of the through-hole TED is aligned with the second hole of the PCB. In some embodiments, the through-hole TED is mounted in the mount area such that the first side is disposed in the second hole. In some embodiments, the through-hole TED is mounted to the PCB such that a plane of the first side of the through-hole TED is substantially parallel to a plane of a second hole. In some embodiments, the PCB comprises a plurality of mount areas, in which each mount area of the plurality comprises the first holes and the second hole. In some embodiments, the apparatus further comprises a plurality of the through-hole TED each mounted in a mount area of the plurality of mount areas, in which each through-hole TED is mounted to a corresponding mount area of the plurality of mount areas with through-hole pins of the through-hole TED attached to the first holes of the corresponding mount area such that the first side of the through-hole TED is positioned to correspond to the second hole of the corresponding mount area.

In some embodiments, the first holes of the mount area comprise one hole and another hole, the one hole and the other hole each comprising a conductive material (e.g., silver, gold, brass, bronze, copper, aluminum, tin, and alloys thereof). In some embodiments, the through-hole TED comprises a second side opposite the first side. In some embodiments, the through-hole TED is operable to heat the first side and cool the second side, or cool the first side and cool the second side, depending on a direction of current applied to the through-hole leads of the through-hole TED. In some embodiments, the apparatus further comprises a first conductive trace connected to the conductive material of the one hole and a second conductive trace connected to the conductive material of the other hole. In some embodiments, the apparatus further comprises at least one circuit to drive current to the one hole and the other hole in a direction to operate the through-hole TED to heat the first side and cool the second side or to cool the first side and cool the second side. In some embodiments, the apparatus further comprises a through-hole TED, in which one lead of the through-hole leads is attached to the one hole of the mount area and another lead of the through-hole leads is attached to the other hole of the mount area.

According to some aspects, apparatus are provided that comprise a printed circuit board (PCB) and a through-hole TED. In some embodiments, the PCB comprises a mount area arranged to have mounted thereon a through-hole thermoelectric device (TED). In some embodiments, the through-hole TED is packaged with a through-hole mount packaging. In some embodiments, the mount area comprises first holes corresponding to through-hole leads of the through-hole TED and a second hole having a size matching a first surface area of a first side of the through-hole TED. In some embodiments, the through-hole TED is mounted in the mount area of the PCB. In some embodiments, the through-hole TED is mounted to the PCB with through-hole pins of the through-hole TED attached to the first holes of the PCB such that the first side of the through-hole TED is aligned with the second hole of the PCB.

In some embodiments, methods are provided for manufacturing a printed circuit board (PCB). In some embodiments, the methods comprise forming the PCB with a mount area arranged to have mounted thereon a through-hole thermoelectric device (TED), in which the through-hole TED is packaged with a through-hole mount packaging, and in which forming the PCB with the mount area comprises forming the PCB with first holes corresponding to through-hole leads of the through-hole TED and/or forming the PCB with a second hole having a size matching a first surface area of a first side of the through-hole TED.

In some embodiments, the step(s) of forming the PCB with the first holes and/or the second hole comprises excising material from the PCB to form the first holes and/or the second hole. In some embodiments, the step(s) of forming the PCB with the first holes and/or the second hole comprises molding the PCB with the first holes and/or the second hole. In some embodiments, the step of forming the PCB with the mount area comprises forming the PCB with a plurality of the mount area, each mount area of the plurality comprising the first holes and the second hole. In some embodiments, the step of forming is performed by manufacturing equipment configured to perform the forming. In some embodiments, the through-hole TED is a thermoelectric cooler (TEC) packaged with the through-hole mount packaging. In some embodiments, the through-hole TED is arranged to create a temperature differential between the first side of the through-hole TED and a second side of the through-hole TED based on a power applied to the through-hole TED. In some embodiments, the through-hole TED is arranged to create a thermal gradient in the through-hole TED via a Peltier effect. In some embodiments, the through-hole TED is operable to adjust a temperature of the first side of the through-hole TED in response to application of power to the through-hole leads.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic drawing of a nucleic acid library preparation workflow;

FIG. 2A is a drawing of a system for automated nucleic acid library preparation using a microfluidic cartridge;

FIG. 2B is a drawing showing internal components of a system for automated nucleic acid library preparation using a microfluidic cartridge;

FIG. 3A is a perspective view of a microfluidic cartridge bay assembly comprising a cartridge;

FIG. 3B is an exploded perspective view of a microfluidic cartridge bay assembly;

FIG. 4A is a top view of a microfluidic cartridge carrier assembly;

FIG. 4B is a perspective view of a microfluidic cartridge;

FIG. 5 is an exploded view of a microfluidic cartridge;

FIG. 6A is a cross-sectional view of a microfluidic cartridge showing two reaction vessels;

FIG. 6B is a perspective view of a base assembly comprising plurality of receptacles;

FIG. 6C is a perspective view of a receptacle having a lock-and-key arrangement with a elongate member comprising a fluidic orifice for interfacing with a reaction vessel;

FIG. 7A is a perspective view of a heated lid;

FIG. 7B is a perspective view of a microfluidic cartridge bay assembly comprising a dual-zoned heated lid;

FIG. 8A depicts an illustrative example of a printed circuit board (PCB) with multiple thermoelectric devices attached at mount areas of the PCB;

FIG. 8B depicts an example of a thermoelectric device with which some embodiments may operate;

FIG. 9A depicts an illustrative example of a PCB having conductive traces and mount areas;

FIGS. 9B and 9C illustrate in more detail an example of a mount area of a PCB with which some embodiments may operate

FIG. 10 illustrates examples of drive electronics for driving a thermoelectric device, that may be implemented in some embodiments; and

FIG. 11 is a flowchart of an illustrative process for mounting thermoelectric devices to a PCB in accordance with some embodiments.

### DETAILED DESCRIPTION

Systems provided herein include thermal control components (e.g., independently thermally controlled receptacles, heated lids, etc.) that function to control the temperature of reaction vessels present in microfluidic cartridges configured for processing molecules, such as nucleic acids. In some embodiments, systems and related methods are provided for automated processing of nucleic acids to produce material (e.g., nucleic acid libraries) for next generation sequencing and/or other downstream analytical techniques. As discussed, systems and apparatus provided herein (e.g., for purposes of preparing a nucleic acid library) may be configured to adjust a temperature of a sample during a process (e.g., a library preparation process). As should be appreciated from the disclosure, a device may be configured in any suitable manner to adjust a temperature. In some embodiments, the device may be configured to adjust the temperature using one or more thermoelectric devices. Such thermoelectric devices may make use of the Peltier effect to cause an exterior surface of the thermoelectric device to be heated and/or cooled, dependent on a direction in which current flows through the thermoelectric device. The thermoelectric devices may act to create a temperature gradient within the device, between a surface that is heated (or cooled) and a surface that is cooled (or heated). An example of such a known thermoelectric device is a thermoelectric cooler (TEC). Those skilled in the art will understand that though the device is termed a thermoelectric “cooler,” it can be equally operated to heat or cool.

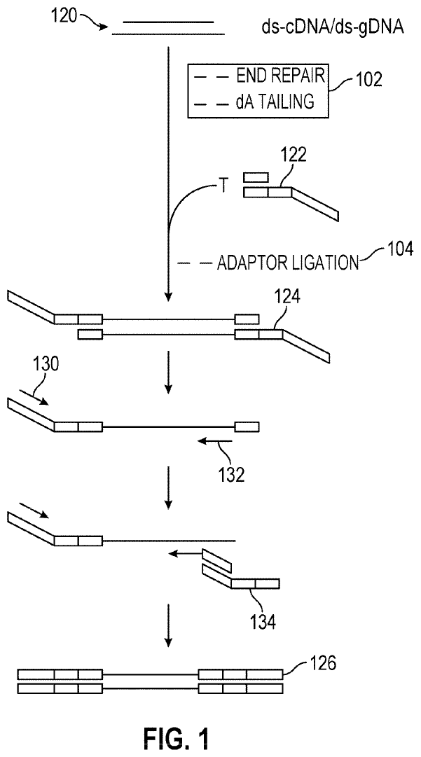

In some embodiments, systems or devices described herein include a cartridge comprising, a frame, one or more cassettes which may be inserted into the frame, and a channel system for transporting fluids. In certain embodiments, the one or more cassettes comprise one or more reservoirs or vessels configured to contain and/or receive a fluid (e.g., a stored reagent, a sample). In some cases, the stored reagent may include one or more lyospheres. The systems and methods described herein may be useful for performing chemical and/or biological reactions including reactions for nucleic acid processing, including polymerase chain reactions (PCR). In some embodiments, systems and methods provided herein may be used for processing nucleic acids as depicted in FIG. 1. For example, in some embodiments, the nucleic acid preparation methods depicted in FIG. 1, which are described in greater detail herein, may be conducted in a multiplex fashion with multiple different (e.g., up to 8 different) samples being processed in parallel in an automated fashion. Such systems and methods may be implemented within a laboratory, clinical (e.g., hospital), or research setting.

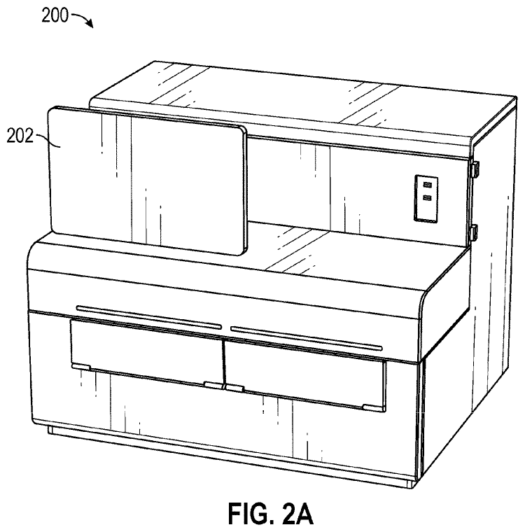

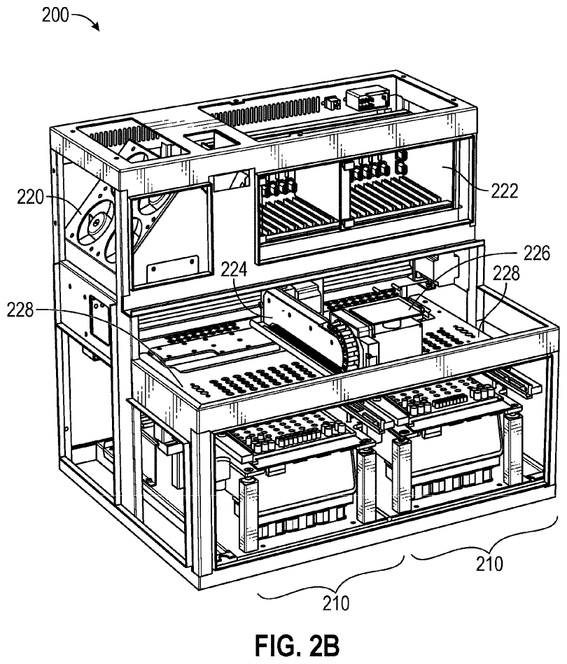

In some embodiments, systems provided herein comprising thermal control components may be used for next generation sequencing (NGS) sample preparation (e.g., library sample preparation). In some embodiments, systems provided herein may be used for sample quality control. FIGS. 2A and 2B depict an example system 200 which serves as a laboratory bench top instrument which utilizes a number of disposable cassettes, primer cassettes, and bulk fluid cassettes. In some embodiments, this system is suitable for use on a standard laboratory workbench.

In some embodiments, a system comprising thermal control components disclosed herein may have a touch screen interface (e.g., as depicted in the exemplary system of FIG. 2A comprising a touch screen interface 202). In some embodiments, the interface displays the status of each of the one or more cartridge bays with “estimated time to complete”, “current process step”, or other indicators. In some embodiments, a log file or report may be created for each of the one or more cartridges. In some embodiments, the log file or report may be saved on the instrument. In some embodiments, a text file or output may be sent from the instrument, e.g., for a date range of cartridges processed or for a cartridge with a particular serial number.

In some embodiments, systems provided herein may comprise one or more cartridge bays (e.g., two, as depicted in the exemplary system of FIG. 2B comprising two cartridge bays 210), capable of receiving one or more nucleic acid preparation cartridges. In some embodiments, a space above the cartridge bay(s) is reserved for an XY positioner 224 to move an optics module 226 (and/or a barcode scanner, e.g., a 2-D barcode scanner) above lids 228 (e.g., heated lids) of each cartridge bay. In some embodiments, the system comprises an electronics module 222 that drives optics module 226 and XY positioner 224. In some embodiments, XY positioner 224 will position optics module 226 such that it can excite materials (e.g., fluorophores) in the vessel and collect the emitted fluorescent light. In some embodiments, this will occur through holes placed in the lid (e.g., heated lid) over each vessel. In some embodiments, a barcode scanner will confirm that appropriate cartridge and primer cassettes have been inserted in the system. In some embodiments, optics module 226 will collect light signals from each cartridge in each cartridge bay, as needed, during processing of a sample, e.g., during amplification of a nucleic acid to detect the level of the amplified nucleic acid. In some embodiments, the systems described herein comprise elements that assist in temperature regulation of components within the system, such as one or more fans or fan assemblies (e.g., the fan assembly 220 depicted in FIG. 2B).

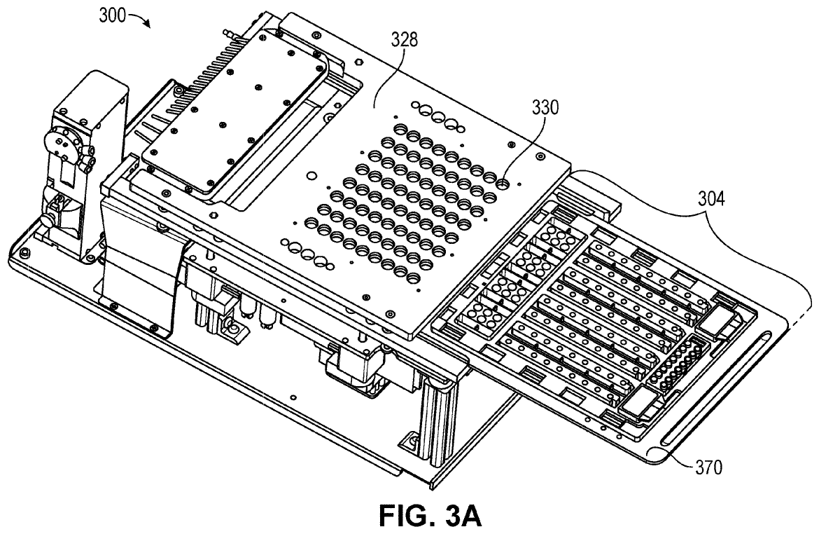

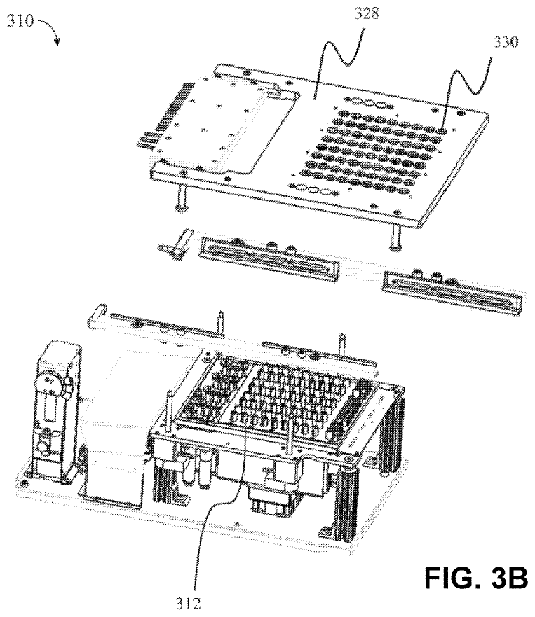

In some embodiments, the one or more cartridge bays can process nucleic acid preparation cartridges, in any combination. In some embodiments, each cartridge bay is loaded, e.g., by the operator or by a robotic assembly. FIGS. 3A and 3B depict exemplary drawings of a microfluidics cartridge bay assembly. FIG. 3A depicts a perspective view 300 of a microfluidic cartridge bay assembly comprising a cartridge. As shown, in some embodiments, a cartridge is loaded into a bay when the bay is in the open position by placing the cartridge into a carrier plate 370 to form a carrier plate assembly 304. The carrier plate is itself, in some embodiments, a stand-alone component which may be removed from the cartridge bay. This cartridge bay holds the cartridge in a known position relative to the instrument. FIG. 3B depicts an exploded perspective view 310 of a microfluidic cartridge bay assembly. As shown, in some embodiments, a lid 328 (e.g., a heated lid) comprises one or more holes 330 to facilitate the processing and/or monitoring of reactions occurring in one or more vessels, each vessel positionable within a thermal jacket 312. In some embodiments, prior to loading a new cartridge onto the instrument, a primer cassette may be installed onto the cartridge. In some embodiments, the primer cassette would be packaged separately from the cartridge. In some embodiments, a primer cassette may be placed into a cartridge. In some embodiments, both primer cassettes and cartridges would be identified such that placing them onto the instrument allows the instrument to read them (e.g., using a barcode scanner) and initiate a protocol associated with the cassettes.

In some embodiments, prior to installing a carrier into the instrument, bulk reagents may be loaded into the carrier. In some embodiments, a user or robotic assembly may be informed as to which reagents to load and where to load them by the instrument or an interface on a remote sample loading station. In some embodiments, after loading a cartridge with a primer cassette into an instrument, a user would have the option of choosing certain reaction conditions (e.g., a number of PCR cycles) and/or the quantity of samples to be run on the cartridge. In some embodiments, each cartridge may have a capacity of 1, 2, 3, 4, 5, 6, 7, 8, 9, 10 or more samples.

In some embodiments, systems provided herein may be configured to process RNA. However, in some embodiments, the system may be configured to process DNA. In some embodiments, different nucleic acids may be processed in series or in parallel within the system. In some embodiments, cartridges may be used to perform gene fusion assays in an automated fashion, for example, to detect genetic alterations in ALK, RET, or ROS1. Such assays are disclosed herein as well as in US Patent Application Publication Number US 2013/0303461, which was published on Nov. 14, 2013, US Patent Application Publication Number and US 2015/02011050, which was published on Jul. 20, 2013, the contents of each of which are incorporated herein by reference in their entirety. In some embodiments, systems provided herein can process in an automated fashion an Xgen protocol from Integrated DNA Technologies or other similar nucleic acid processing protocol.

In some embodiments, cartridge and cassettes will have all of the reagents needed for carrying out a particular protocol. In some embodiments, once a carrier is loaded into a cartridge bay an access door to that bay is closed, and optionally a lid (e.g., a heated lid) may be lowered automatically. In some embodiments, lowering of the lid (e.g., the heated lid) forces (or places) the cartridge down onto an array of heater jackets which conform to each of a set of one or more temperature controlled vessels in the cartridge. In some embodiments, this places the cartridge in a known position vertically in the drawer assembly. In some embodiments, lowering of the lid forces the cartridge down into a position in which rotary valves present in the cartridge are capable of engaging with corresponding drivers that control the rotational position of the valves in the cartridge. In some embodiments, automation components are provided to ensure that the rotary valves properly engage with their drivers.

In some embodiments of methods provided herein, a nucleic acid sample present in a cartridge (e.g., within a vessel of a cassette) will be mixed with a lyosphere. In some embodiments, the lyosphere will contain a fluorophore which will attach to the sample. In some embodiments, there will also be a “reference material” in the lyosphere which will contain a known amount of a molecule (e.g., of synthetic DNA). In some embodiments, attached to the “reference material” will be another fluorophore which will emit light at a different wavelength than the sample's fluorophore. In some embodiments, fluorophores used may be attached to the sample or the “reference material” via an intercalating dye (e.g., SYBR Green) or a reporter/quencher chemistry (e.g., TaqMan, etc.). In some embodiments, during quantitative PCR (qPCR) cycling the fluorescence of the two fluorophores will be monitored and then used to determine the amount of nucleic acid (e.g., DNA, cDNA) in the sample by the Comparative CT method.

Advantageously, certain systems described herein may include modular components (e.g., cassettes) that can allow tailoring of specific reactions and/or steps to be performed. In some embodiments, certain cassettes for performing a particular type of reaction are included in the cartridge. For example, cassettes including vessels containing lyospheres with different reagents for performing multiple steps of a PCR reaction may be present in the cartridge. The frame or cartridge may further include empty regions for a user to insert one or more cassettes containing specific fluids and/or reagents for a specific reaction (or set of reactions) to be performed in the cartridge. For example, a user may insert one or more cassettes containing particular buffers, reagents, alcohols, and/or primers into the frame or cartridge. Alternatively, a user may insert a different set of cassettes including a different set of fluids and/or reagents into the empty regions of the frame or cassette for performing a different reaction and/or experiment. After the cassettes are inserted into the frame or cartridge, they may form a fluidic connection with a channel system for transporting fluids to conduct the reactions/analyses.

In some embodiments, multiple analyses may be performed simultaneously or sequentially by inserting different cassettes into the cartridge. For instance, the systems and methods described herein may advantageously provide the ability to analyze two or more samples without the need to open the system or change the cartridge. For example, in some cases, one or more reactions with one or more samples may be conducted in parallel (e.g., conducting two or more PCR reactions in parallel). Such modularity and flexibility may allow for the analysis of multiple samples, each of which may require one or several reaction steps within a single fluidic system. Accordingly, multiple complex reactions and analyses may be performed using the systems and methods described herein.

Unlike certain existing fluidic systems and methods, the systems and methods described herein may be reusable (e.g., a reusable carrier plate) or disposable (e.g., consumable components including cassettes and various fluidic components). In some cases, the systems described herein may occupy a relatively small footprint as compared to certain existing fluidic systems for performing similar reactions and experiments.

In some embodiments, the cassettes and/or cartridge includes stored fluids and/or reagents needed to perform a particular reaction or analysis (or set of reactions or analyses) with one or more samples. Examples of cassettes include, but are not limited to, reagent cassettes, primer cassettes, buffer cassettes, waste cassettes, sample cassettes, and output cassettes. Other appropriate modules or cassettes may be used. Such cassettes may be configured in a manner that prevents or eliminates contamination or loss of the stored reagents prior to the use of those reagents. Other advantages are described in more detail below.

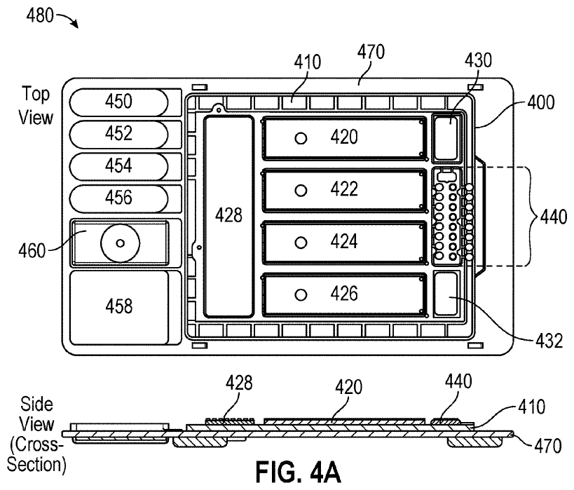

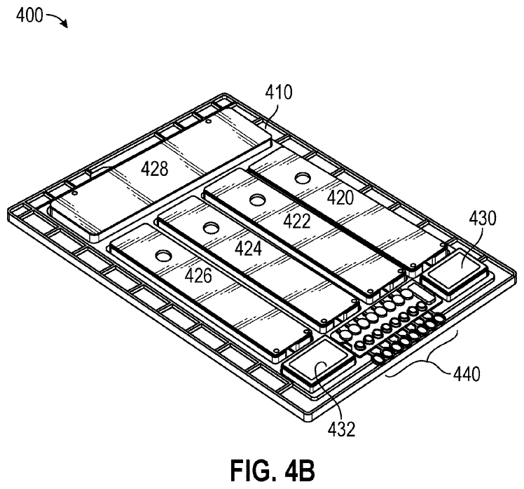

In one embodiment, as shown illustratively in FIGS. 4A and 4B, cartridge 400 comprises a frame 410 and cassettes 420, 422, 424, 426, 428, 430, 432, and 440. In some embodiments, each of these cassettes may be in fluidic communication with a channel system (e.g., positioned underneath the cassettes, not shown). In some embodiments, at least one of cassettes 428 (e.g., a reagent cassettes), 430 (e.g., a reagent cassette), and 432 (e.g., a reagent cassette) may be inserted into frame 410 by the user such that the cassettes are in fluidic communication with the channel system. For example, in some embodiments, one of cassettes 428, 430, and 432 is a reagent cassette containing a reaction buffer (e.g., Tris buffer). In certain embodiments, cassettes 428, 430 and/or 432 may comprise one or more reagents and/or reaction vessels for a reaction or a set of reactions. In some embodiments, module 440 comprises a plurality of sample wells and/or output wells (e.g., samples wells configured to receive one or more samples). In some cases, cassettes 420, 422, 424, and 426 may comprise one or more stored reagents or reactants (e.g., lyospheres). For instance, each of cassettes 420, 422, 424, and 426 may include different sets of stored reagents or reactants for performing separate reactions. For example, cassette 420 may include a first set of reagents for performing a first PCR reaction, and cassette 422 may include a second set of reagents for performing a second PCR reaction. The first and second reactions may be performed simultaneously (e.g., in parallel) or sequentially.

In some embodiments, as shown illustratively in FIG. 4A, a carrier plate assembly 480 comprises a carrier plate 470 and additional cassettes including modules 450, 452, 454, 456, 458, and 460. In an exemplary embodiment, cassettes 450, 452, 454, 456, 458, and 460 may each comprise one or more stored reagents and/or may be configured and arranged to receive one or more fluids (e.g., module 458 may be a waste module configured to collect reaction waste fluids). In some embodiments, one or more of cassettes 450, 452, 454, 456, 458, and 460 may be refillable.

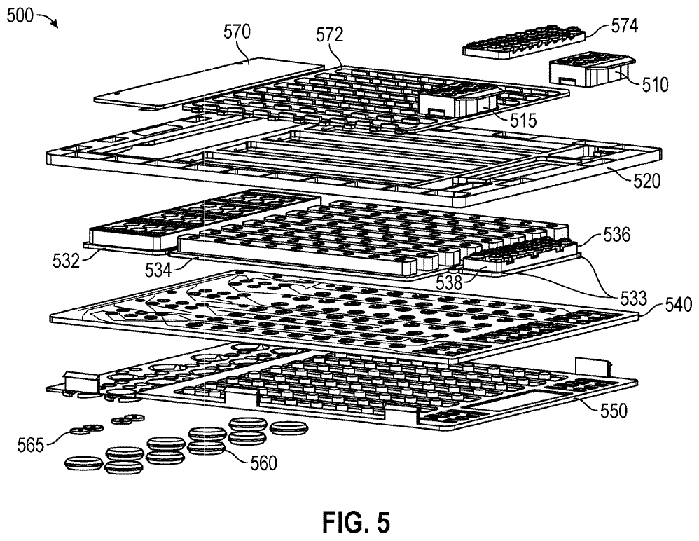

FIG. 5 is an exploded view of an exemplary cartridge 500, according to one set of embodiments. Cartridge 500 comprises a primer cassette 510 and a primer cassette 515 which may be inserted into one or more openings in a frame 520. Cartridge 500 further comprises a fluidics layer assembly 540 containing a channel system adjacent and non-integral to frame 520. In some embodiments, a set of cassettes 532 (e.g., comprising one or more primer cassettes, buffer cassettes, reagent cassettes, and/or waste cassettes, each optionally including one or more vessels), set of reaction cassettes 534, which comprises reaction vessels, an input/output cassette 533, which comprises sample input vessels 536 and output vessels 538, may be inserted into one or more openings in frame 520. In some embodiments, cartridge 500 comprises a valve plate 550. In some embodiments, valve plate 550 connects (e.g., snaps) into frame 520 and holds in place fluidics layer assembly 540 and cassettes 532, 533 and 534 in frame 520. In certain embodiments, cartridge 500 comprises valves 560, as described herein, and a plurality of seals 565. In some cases, frame 520 and/or one or more modules may be covered by covers 570, 572, and/or 574.

Apparatus are also provided for independently manipulating the temperature of a plurality of reaction vessels that include a base assembly comprising a plurality of receptacles (e.g., 6, 12, 24, 36, 64, 96, 384 thermoelectric devices). Each receptacle may be configured to have disposed therein a reaction vessel. In some embodiments, the base assembly further comprises a plurality of thermoelectric devices (e.g., 6, 12, 24, 36, 64, 96, 384 thermoelectric devices) configured to heat or cool each receptacle/reaction vessel.

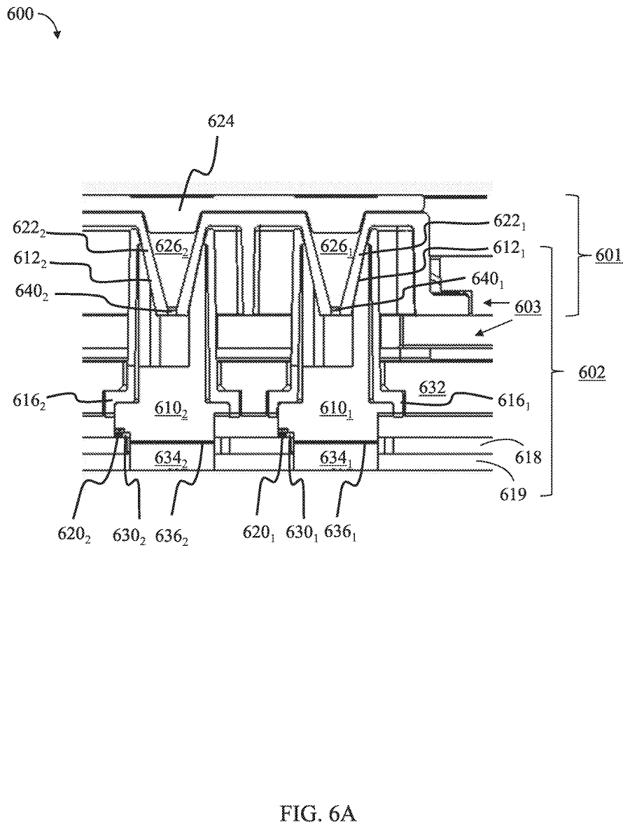

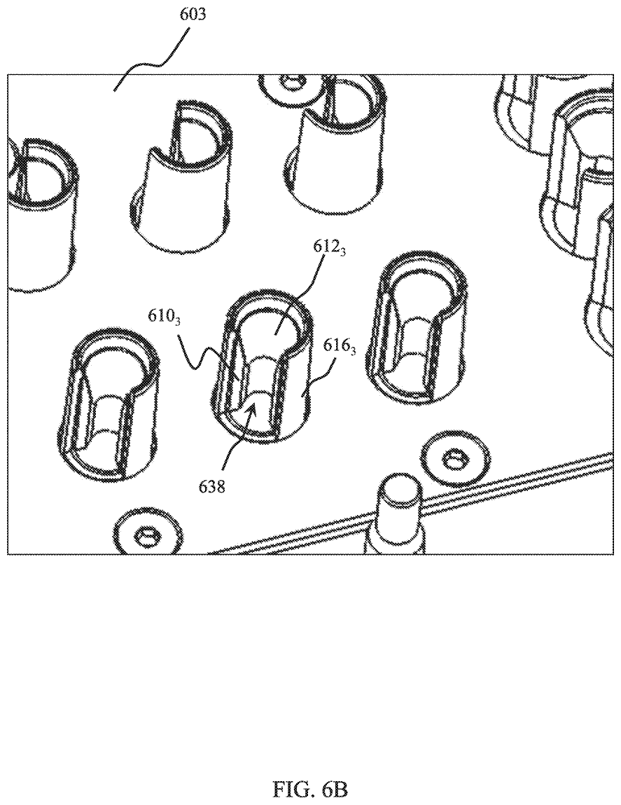

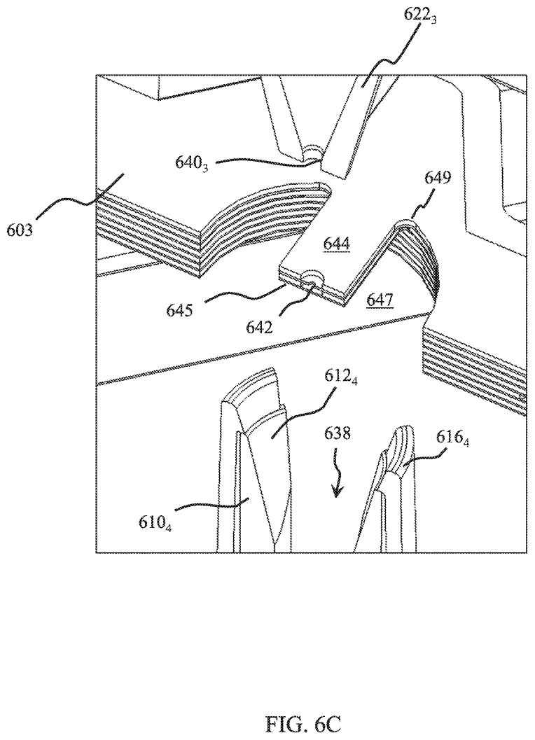

FIGS. 6A-6C provide non-limiting examples of apparatus 600 having reaction vessels 622₁₋₃ of a cassette assembly 601 and corresponding receptacles 626₁₋₂ of a base assembly 602. FIG. 6A is a cross-sectional view of two reaction vessels 622₁₋₂ of a cassette assembly 601 disposed in corresponding receptacles 626₁₋₂ of a base assembly 602. Each receptacle 626₁₋₂ comprises a thermal jacket 610₁₋₂ in thermal communication a thermoelectric device 634₁₋₂. The thermal jacket 610₁₋₂ has a first thermal transfer surface 612₁₋₂ configured to surround at least a portion of a reaction vessel 622₁₋₂ to facilitate thermal exchange between the reaction vessel 622₁₋₂ and thermal jacket 610₁₋₂. In some embodiments, a first thermal transfer surface 612₁₋₂ configured contacts the reaction vessel 622₁₋₂ and/or surrounds a portion of the outer circumference of the reaction vessel 622₁₋₂. The thermal jacket 610₁₋₂ may have a second thermal transfer surface 636₁₋₂ that interfaces with the thermoelectric device 634₁₋₂ to facilitate heat exchange between the thermal jacket 610₁₋₂ and the thermoelectric device 634₁₋₂. The second thermal transfer surface 636₁₋₂ is in contact with a thermal pad 630₁₋₂ that interfaces with the thermoelectric device 634₁₋₂ to facilitate heat exchange between the thermal jacket 610₁₋₂ and the thermoelectric device 634₁₋₂. In some embodiments, the thermal pad 630₁₋₂ is composed of silver, bronze, brass, aluminum, copper, steel, alloys thereof or other thermally conductive material. In some embodiments, the thermal pad 630₁₋₂ has a thermal resistance (Modified ASTM D5470) in a range of 3° C.-cm²/watt to 6° C.-cm²/watt. In some embodiments, a reaction vessel may comprise an inlet port 640₁₋₂ at the base of a reaction vessel 622₁₋₂ to permit flow of fluid into and out from the reaction vessel 622₁₋₂.

In some embodiments, the thermoelectric device 634₁₋₂ is a solid-state heat pump that operates according to a Peltier effect, in which heating or cooling occurs when electric current passes through two conductors comprising dissimilar materials. In some embodiments, a voltage applied to the free ends of two dissimilar materials creates a temperature difference, such that heat moves from one end to the other. In some embodiments, the thermoelectric device 634₁₋₂ comprises an array of p- and n-type semiconductor elements that act as the two dissimilar conductors. In some embodiments, the array of elements is joined or soldered between two plates (e.g., ceramic plates) electrically in series and thermally in parallel. In some embodiments, a current (DC current) passes through one or more pairs of elements from n- to p-, there is a decrease in temperature at the junction, creating a cool side and resulting in the absorption of heat from the environment. In some embodiments, the absorbed heat transfers through the device and is released on the opposite side as electrons move from a high- to low-energy state. In some embodiments, the heat-pumping capacity of the thermoelectric device 634₁₋₂ is proportional to the current and the number of pairs of n- and p-type elements (or couples). In some embodiments, n- and p-type semiconductors (usually Bismuth Telluride) are preferred materials used to achieve a Peltier effect because they can be easily optimized for pumping heat. In some embodiments, can also control the type of charge carrier within the conductor. In some embodiments, the thermoelectric device 634₁₋₂ is a Peltier device configured to operate as a heat source or heat sink for the thermal jacket 610₁₋₂. In some embodiments, the thermoelectric devices are Peltier devices configured to cycle between heating and cooling operations relative to the thermal jacket 610₁₋₂. The thermal jacket 610₁₋₂ is also surrounded by a thermal insulator 616₁₋₂ that contains heat within the thermal jacket 610₁₋₂. A base plate 632 (e.g., which may be composed of aluminum) is provided that holds the thermal jacket 610₁₋₂ and thermal insulator 616₁₋₂ in place against a printed circuit board (PCB) 618 which is sandwiched between the base plate 632 and a foam base 619 (e.g., which may be composed of silicon). The thermoelectric devices 634₁₋₂ are mounted on the printed circuit board (PCB) 618, as described further herein. One or more thermistors 620₁₋₂ are provided that monitor temperature of the thermoelectric devices 634₁₋₂ base on thermally dependent changes in resistance and enables feedback control of the heating or cooling effect produced by the thermoelectric devices 634₁₋₂.

An apparatus may comprise a cassette assembly 601 comprising a plurality of reaction vessels 622₁₋₂. In some embodiments, the cassette assembly 601 is configured and arranged to interface with the base assembly 602 such that each of the plurality of receptacles 626₁₋₂ of the base assembly 602 has disposed therein a corresponding reaction vessel 622₁₋₂ of the cassette assembly 601. In some embodiments, the apparatus further comprise a plurality of cassette assemblies 601, each cassette assembly 601 comprising a plurality of reaction vessels 622₁₋₂. In some embodiments, each cassette assembly 601 is configured and arranged to interface with the base assembly 602 such that each of the plurality of receptacles 626₁₋₂ of the base assembly 602 has disposed therein a corresponding reaction vessel 622₁₋₂ of the cassette assembly 601. In some embodiments, the cassette assemblies 601 are disposed in a cartridge (such as that depicted in FIG. 5).

FIG. 6B shows a cross-sectional perspective view of a portion of cartridge comprising a support structure 603 comprising a channel apparatus having a plurality of fluidic conduits. The support structure 603 comprises a plurality of layers (referred to as a fluidics layer assembly) within which several fluidics passages are present that direct the flow of fluid to and from different locations within the assembly. For example, fluidics passages are provided within the layer assembly that direct flow of fluid from one or more reagent cassettes to one or more reaction vessels and/or one or more reaction vessels to one or more other cassettes. Thus, in some embodiments, one or more fluidic conduits is in fluidic communication with a reaction vessel. FIG. 6B shows a base assembly comprises a plurality of receptacles 612₃ each having a thermal jacket 610₃ surrounded by a thermal insulator 616₃.

The receptacles have a lock and key feature in which a keyway 638 permits an elongate member of a fluidics layer assembly to pass into the receptacle together with a reaction vessel. The elongate member may comprise a channel that fluidically interface with the base of the reaction vessel to permit fluidic exchange between the reaction vessel and other components of the system while the vessel is present inside the receptacle.

As depicted in FIG. 6C a fluidic conduit may end at a fluid outlet orifice 642 that fluidically interfaces with an inlet port 640₃ at the base of a reaction vessel 622₃ to permit flow of fluid into and out from the reaction vessel 622₃. The support structure 603 may be configured with a plurality of openings 647 aligned with one or more reaction vessels 622₃. In some embodiments, each opening is configured to permit passage of a thermal jacket 610₄ through the support structure 603 to access and surround the reaction vessel 622₃. In some embodiments, the support structure 603 comprises a plurality of elongate members 644, in which each elongate member extends from an outer edge 649 of an opening 647 to an inner position 645 of the opening 647. In some embodiments, a fluidic conduit of a fluidics layer assembly extends through the elongate member 644 from the outer edge 649 to the inner position 645 ending at a fluid outlet orifice 642 at the inner position 645. In some embodiments, the thermal jacket has a keyway 638 that aligns with the elongate member 644 to permits passage of the thermal jacket 610₄ through the opening 647 to access and surround the thermal jacket 610₄. The thermal jacket 610₄ has a first thermal transfer surface 612₄ and is surrounded by a thermal insulator 616₄ that contains heat within the thermal jacket 610₄.

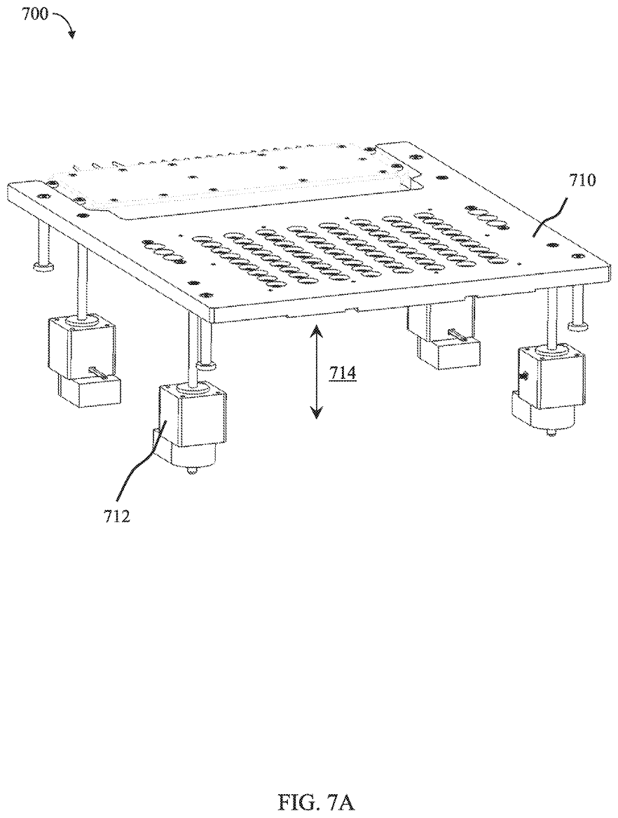

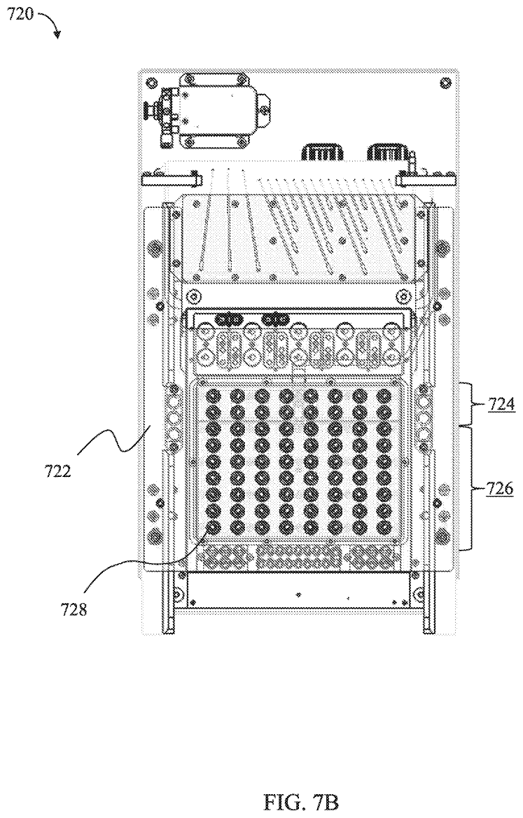

According to certain aspects of the invention, apparatus are provided for managing temperature of one or more reaction vessels. As depicted in FIGS. 7A and 7B, the apparatus may comprises a thermal cover assembly 700. In some embodiments, the thermal cover assembly comprises a thermal cover (or lid) 710 that is configured to provide a heat source above each reaction vessel to prevent or minimize evaporation of a reaction solvent present in the reaction vessel. The thermal cover assembly is typically fitted within cartridge bay assembly and includes one or more clamping motors 712 that control the vertical position 714 of the thermal cover. For example, when a cartridge is inserted into a bay assembly the clamping motors 712 may be activate to clamp the thermal cover assembly down against the cartridge and compressing the cartridge against a base assembly such that reaction vessels present in the cartridge are position down into corresponding receptacles of the base assembly. FIG. 7B provides a top view of bay assembly 720 showing transparently a thermal cover 722, the thermal cover assembly has at least two independently thermally controlled zones (e.g., 2, 3, 4, 5, 6, 7, 8, 9, 10 or more zones). In some embodiments, the thermal cover assembly has two independently thermally controlled zones, a first zone 724 providing an independently controlled heat source or sink above a set of sixteen reaction vessels 728, and a second zone 726 providing an independently controlled heat source or sink above a set of fifty-six reaction vessels. The thermal cover assembly is configured with a plurality of openings to permit the passage of light into and out from the reaction vessel, thereby permitting optical measurements from each reaction vessel.

Described herein are examples of ways in which thermoelectric devices may be arranged in a system, apparatus or device (e.g., a library preparation device) to adjust a temperature of a vessel or sample therein. It should be appreciated, however, that embodiments are not limited to being implemented in accordance with embodiments described below, as other embodiments are possible.

Those skilled in the art will appreciate that known TECs are not packaged to be surface-mounted to a printed circuit board. Rather, known TECs are packaged with terminal wires that, in the context of PCB dimensions, may be considered lengthy. In some embodiments, these terminal wires permit control electronics for the TECs to be disposed remote from the TEC and remote from the environment that the TEC is to heat and/or cool. This is advantageous, as in many use cases the element to be heated or cooled may have a significantly high temperature that could damage control electronics proximate to such a high temperature. For example, an element to produce laser light may produce a substantial amount of heat and a TEC may be arranged to sink that heat. Using long terminal wires, the control electronics may be disposed remote from that heat.

Such TECs with lengthy terminal wires may be referred to as having “cable mount” packaging, to distinguish them from other devices with other types of packaging. Such “cable mount” packaging is not used with printed circuit boards (PCBs) or other forms of surface mounting of electrical components. Rather, electrical components to be mounted to PCBs use different types of packaging, often referred to as surface mount packaging.

The inventors have recognized and appreciated that TECs perform a heating/cooling function and as such might be used in a library preparation device for heating and/or cooling a sample during library preparation. The inventors have also recognized and appreciated, however, that cable-mount TECs are disadvantageous for use with a library preparation device. In some embodiments, it is advantageous to mount TEC's on a PCB to avoid difficulties with cable management. In some embodiments, precise temperature control is important to process performance, and TEC's allow variation based on individual component performance, so direct temperature monitoring with local thermistors is aided with mounting both to the PCB, and preforming subsequent calibrations. As discussed above, it is advantageous for wells holding biological samples in a library preparation device to be individually controlled to be heated or cooled. If multiple TECs were to individually correspond to multiple wells to provide such individual control, the terminal wires of cable-mount TECs would provide a challenge, as the terminal wires would need to be routed to each TEC and would likely obstruct other components of the library preparation device that are to be placed proximate the wells that hold biological samples (e.g., other components to process the biological samples in other ways).

Moreover, TECs are adapted for use with a surface of the TEC applied or proximate to an element to be heated or cooled. As such, any component to which the TECs are mounted would need to support the TECs being disposed such that a surface of each TEC is proximate to the well to be individually heated/cooled. Arranging multiple TECs so as to individually contact a well would provide a number of challenges, including in reliably mounting the TECs.

The inventors have additionally recognized and appreciated, however, that a printed circuit board (PCB) may be adapted for use with TECs or other thermoelectric devices, and that TECs may in turn be adapted for use with such a PCB. For example, a printed circuit board may be arranged with mount areas to which each TEC may be mounted. That mount area may include a hole with which a surface of the corresponding TEC is to be aligned, such that a surface of the TEC to be heated or cooled is accessible via the hole. In some such embodiments, the surface of the TEC may be disposed in the hole. In other embodiments, the surface of the TEC may be aligned with the hole with a plane of the TEC substantially parallel to a plane of the hole, though the surface of the TEC may not be in the hole.

In some embodiments, a gap filler pad (e.g., a sheet of TGard, a silicone/boron nitride composite pad, etc.) is provided that is thermally conductive and can be located on either top or bottom side of the TEC or on both sides. In some embodiments, on the opposite side of the TEC there will be a thermally conductive paste. In some embodiments, the thermal pad and/or thermally conductive paste are compressible and used to accommodate variation in clamping pressure and variation in component thicknesses. In some embodiments, a chain of components from heat sink, TEC, and thermal jacket is supplemented with these layers to ensure positive contact for conduction heat transfer instead of slower convective heat transfer which occurs when there is a slight gap.

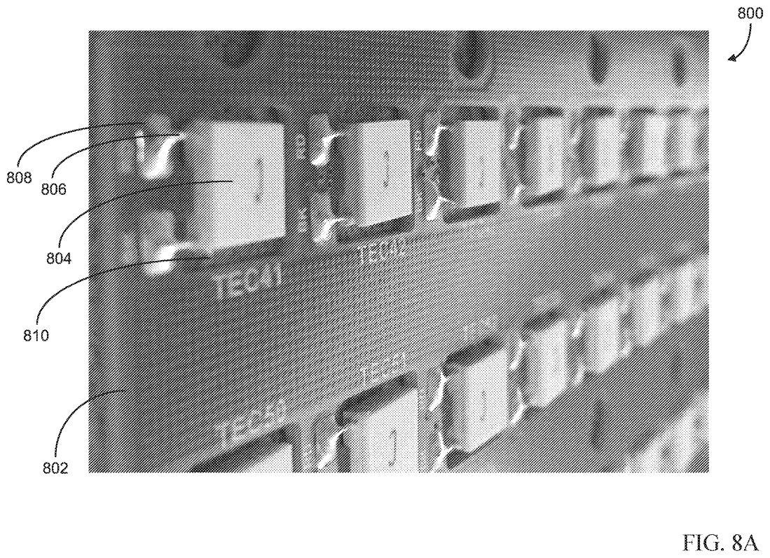

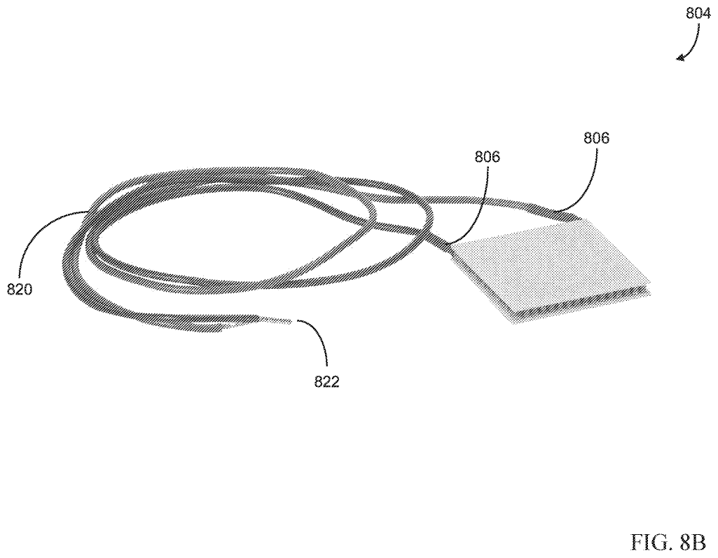

FIG. 8A depicts an example of an apparatus 800 including a printed circuit board 802 having mount areas to which TECs 804 are mounted. As discussed above, each TEC 804 may include terminal wires 806 so as to be cable-mounted, as known TECs are conventionally connected to other components. In accordance with techniques described herein, however, the terminal wires of the TEC may be shortened (or short) for being affixed to a mount pad 808 of a mount area of a PCB. For example, the terminal wires of the TEC may have a length smaller than a length or width of a surface of the TEC to be heated/cooled. The terminal wires of the TEC may have a length smaller than a length or width of a mount area of the PCB to which the TEC is to be mounted, or smaller than a length or width of a hole of the mount area. The terminal wires may have a length similar to a width of a mount pad 808 of the mount area, or a length less than or equal to a distance from an edge of the hole 810 of the mount area to a side of the mount pad of the mount area farthest from the hole. As shown in FIG. 8A, the two mount pads to which the terminal wires 806 are connected may be arranged adjacent to a hole 808 of the mount area. FIG. 8A illustrates multiple TECs and multiple mount areas in the apparatus 800. Additional details of the apparatus 800 and the mount areas are provided below.

For comparison, FIG. 8B illustrates an example of a TEC 804 having conventional packaging. The packaging of the TEC 804 includes two terminal wires 806, with those two terminal wires including lengthy wires 820 having a length much larger than the length or width of the surface of the TEC 804 that is heated or cooled. The terminals of the wires 820 are arranged to be inserted into a socket such as a breadboard socket. Thus, unlike the arrangement of apparatus 800 of FIG. 8A, the TEC 804 of FIG. 8B is arranged to be located distant from (i.e., at the other end of wires 820) a connection point for the terminals 822. As should be appreciated from the photograph of FIG. 8A, such an arrangement would be difficult with multiple TECs in close proximity.

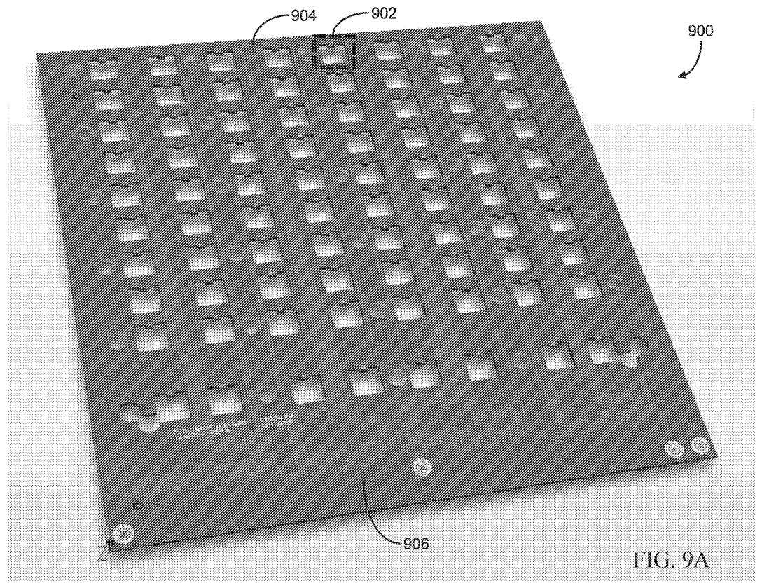

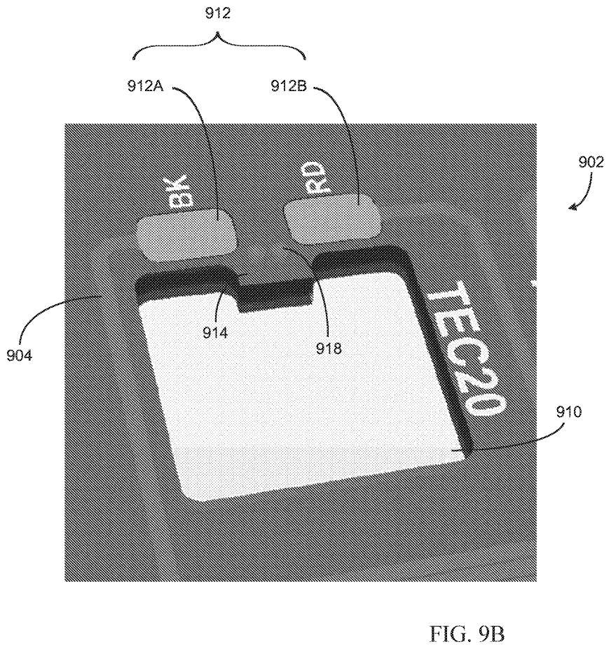

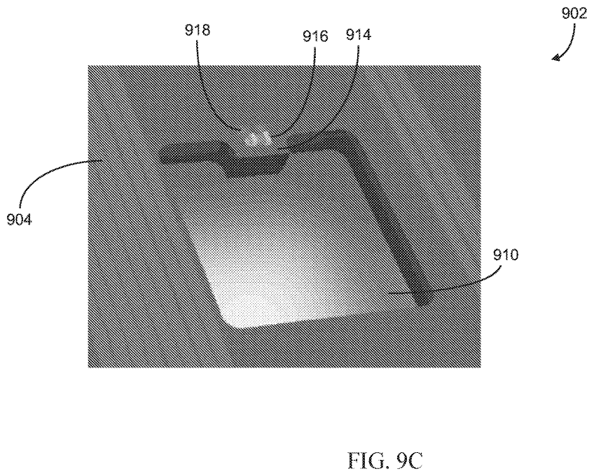

FIG. 9A illustrates a printed circuit board (PCB) having multiple mount areas 902 and conductive traces 904 connecting the mount areas 902 to a control area 906. While not illustrated in FIG. 9A, each mount area 902 is arranged to have a TEC mounted thereto and the control area 906 is arranged to have control electronics to drive the TECs mounted thereto.

The PCB 900 may be produced using any suitable technique, including known techniques. For example, the PCB 900 may include multiple layers of conductive interconnect and laminates, with vias or other conductive structures providing for passages between layers.

As shown in FIG. 9A, the PCB 900 may be formed with multiple mount areas and, thus, multiple holes. For example, the PCB 900 may be formed with more than 5 mount areas, more than 10 mount areas, more than 25 mount areas, or more than 50 mount areas. As mentioned above and as discussed in more detail below, each mount area may include a hole corresponding to a component to be aligned with the hole. Accordingly, the PCB may include more than 5 holes, more than 10 holes, more than 25 holes, or more than 50 holes each corresponding to a component to be aligned with the hole.

The mount areas (and the holes) may be arranged in any suitable manner. For example, as shown in FIG. 9A, the mount areas (and holes) may be arranged according to a regular pattern, such as a two-dimensional array.

FIGS. 9B and 9C provide additional detail on a mount area 902 of the PCB 900 of FIG. 9A. FIG. 9B illustrates the mount area 902 on one side of the PCB 900, while FIG. 9C illustrates the mount area 902 on a second, opposite side of the PCB 900. FIGS. 9B and 9C show a single mount area 902. In some embodiments, however, each mount area of the PCB 900 may be arranged in an identical manner.

As discussed above, mount area 902 includes a hole 910 through all layers of the PCB 900. The hole 910 may have dimensions corresponding to dimensions of a TEC that will be attached to the mount area 902. In some embodiments, dimensions may also be of smaller or irregular shape or simple shape such as a circle, which can also be used as a locator for the thermal jacket. In some embodiments, provided that the thermal jacket and heat sink have access for thermal connection to both sides of the TEC, many configurations and hole size and shapes are possible. Specifically, the hole 910 may have a length and width that corresponds to a length and width of a surface of the TEC that is to be heated and/or cooled. The dimensions may correspond such that the surface of the TEC may be aligned with the hole 910 without the edges of the TEC contacting edges of the hole 910 when properly aligned. To permit for some slight variation in positioning that may occur during manufacturing, the dimensions of the hole 910 may be somewhat larger than dimensions of the surface of the TEC to be heated and/or cooled. For example, the dimensions of the hole 910 may be 5% or 10% larger than dimensions of the surface of the TEC to be heated and/or cooled.

The hole 910 may be formed in any suitable manner. In some embodiments, the PCB 900 may be formed using a mold or guide that includes the hole 910, such that material for layers of the PCB 900 is not deposited in areas in which the holes 910 of the mount area are to be located. In other embodiments, the PCB 900 may be created without the holes 910 and then holes 910 may be subsequently etched, cut, or otherwise excised from the PCB 900.

The mount area 902 additionally includes mount pads 912, individually referred to as mount pad 912A and mount pad 912B. The mount pads may be made of any suitable conductive material that may be used in a print circuit board. In some embodiments, the mount pads 912 may include a gold plating to ensure high conductivity. In the example of FIG. 9B, the mount area 902 is arranged to be used with a TEC having two terminal wires and, as such, two mount pads 912 are provided. Further, the mount area 902 is arranged for use with a TEC having two terminal wires arranged on a same side of the TEC. In other embodiments in which terminal wires are otherwise arranged (e.g., more or less terminal wires or terminal wires on multiple sides of the TEC), the mount pads 912 may be arranged otherwise, such as by including more or less mount pads or having mount pads positioned differently.

Mount area 902 is also connected to one or more conductive traces 904, to permit electrical signals to be provided to or from components attached to the PCB at the mount area 902. The hole 910 may be of a shape substantially conforming to a shape of a surface of a TEC to be aligned with the hole 910. For example, the hole 910 may be substantially rectangular (including substantially square). However, in some embodiments, the hole 910 may include a protrusion 914 that extends from the PCB 900 into the hole 910. The protrusion 914 may be formed of the same laminate or other materials of the PCB 900. For example, the PCB 900 and/or hole 910 may be formed with the protrusion 914.

The protrusion 914 may be located on any side of the hole 910. In embodiments in which two terminal leads of a TEC are located on a same side of the TEC and the mount pads 912 are positioned on a corresponding side of the hole 910 (as is the case in the example of FIG. 9B), the protrusion 914 may extend from the side of the hole 910 on which the mount pads 912 are positioned.

The protrusion 914 may permit a monitoring of temperature created by a TEC that is attached to the mount area 902 and aligned with the hole 910. Specifically, a temperature-monitoring device may be positioned on the protrusion 914, so as to be positioned closer to the surface of the TEC that is heated/cooled and as such better monitor a temperature created by the TEC. FIG. 9C illustrates an example of such a temperature-monitoring device 916 positioned on the protrusion 914. In the example of FIG. 9C, the temperature-monitoring device is a thermistor. Other electrical components or combinations of electrical components may be used, however.

In the example of FIGS. 9B-9C, the thermistor 916 is positioned on a side of the PCB opposite from a side on which the TEC is mounted. The thermistor 916 may be attached to a mount area for the thermistor, which may include one or more conductive mounting pads or terminuses of conductive traces. Positioning the thermistor 916 in this was may allow for the thermistor to better monitor a temperature created by the TEC in the environment of the TEC. This may be because the thermistor is separated from the TEC by the PCB and thus may more accurately monitor a temperature of the environment of the TEC, rather than the TEC itself. In addition, in some embodiments the components or biological samples to be heated or cooled by the TEC may be positioned on a side of the PCB opposite from the side on which the TEC is mounted. By positioning the thermistor 918 on that side (as shown in FIG. 9C), the thermistor may be positioned closer to the components/samples being heated or cooled and obtain a potentially more accurate reading on the temperature imposed on the component or sample by the TEC.

The thermistor 918 may be connected via conductive traces 904 to control electronics that control the TEC, such that the control electronics may control the TEC based on the temperature detected by the thermistor 918. The thermistor 918 may therefore act as part of a feedback loop.

The thermistor 918 may be connected to the conductive traces 904 via one or more vias 918, in some embodiments. The vias 918 are conductive tunnels through one or more layers of the PCB and allow for a component on one side of a PCB (e.g., the thermistor 918) to be connected to a conductive trace 904 at another side or in another layer of the PCB. The vias 918 are shown on both sides of the PCB in FIGS. 9B and 9C, but those skilled in the art will appreciate that a via 918 may not extend fully through a PCB.

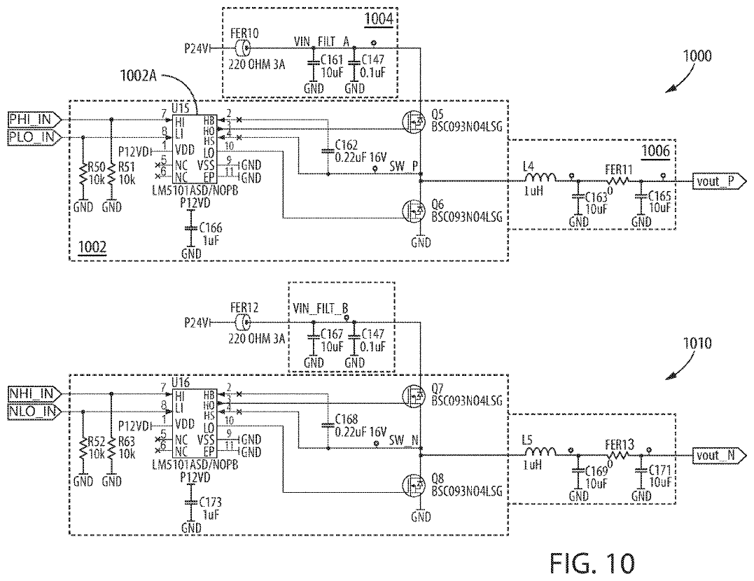

Embodiments are not limited to working with any specific form of control electronics for driving a TEC. FIG. 10 illustrates an example of control electronics that may be connected to a TEC in some embodiments. In particular, circuits 1000, 1010 are examples of circuits that may perform pulse width modulation (PWM) control of a TEC. In an embodiment implementing circuits 1000 and 1010, circuit 1000 may be connected to a positive terminal of a TEC and circuit 1010 may be connected to a negative terminal of a TEC. The circuits are similarly structured and one of ordinary skill in the art should understand how to implement circuit 1010 from the description of circuit 1000. As discussed above, the TEC may be operated to heat or cool a side of the TEC based on a direction of current flow. Circuit 1000 may be used to drive the TEC when a current is to be applied to a positive terminal of the TEC to cool a first side of the TEC (and to correspondingly heat a second side), with current flowing out of the negative terminal to ground. Circuit 1010 may be used to drive the TEC when a current is to be applied to a negative terminal of the TEC to heat the first side of the TEC (and to correspondingly cool the second side), with current flowing out of the positive terminal to ground. Accordingly, control may be switched between circuit 1000 or circuit 1010 based on how the TEC is to be operated.

Circuit 1000 includes a pulse width modulation (PWM) control circuit 1002, including a PWM controller 1002A. In some embodiments, a switching signal/pulse controller is often used for efficient energy consumption. In some embodiments, an analog signal approach may be used by controlling the voltage supplied to each TEC instead of the duty cycle. The pulse width controller 1002A produces a signal having a duty cycle that drives two drive transistors. The signal switches back and forth between “high” and “low,” and when at a high state activates the transistors to provide power to the TEC.

The transistors of PWM control circuit 1002 are connected to a drive voltage for the TECs, which in the example of FIG. 10 is a 24V input.

Some TECs may be arranged to heat or cool based in part on how long they are driven with current through the terminals of the TECs. This may be because there is some delay after a current is imposed before the TEC starts actively heating or cooling one side of the TEC, and because the TEC may stop heating/cooling nearly immediately after current is stopped. Accordingly, these TECs may be sensitive to any variation in a drive signal. A short absence of low state in the signal may significantly affect operation of the TEC, as the TEC may stop heating/cooling in response to the absence or low state. Accordingly, in some such embodiments, the control electronics for the TEC may include filters to eliminate short, high-frequency variations in the signal. A filter 1004, which may be a low-pass filter, is connected to the 24V drive signal input for the TEC. In addition, because of the potential for high-frequency variations in the output of the PWM control circuit 1002, a second low-pass filter 1006 may be connected between the output of the PWM control circuit 1002 and the input to the TEC (at the right side of circuit 1000).

While an example of control electronics using pulse width modulation has been provided, it should be appreciated that in some embodiments different voltage converters may be used to produce a drive signal for a TEC.

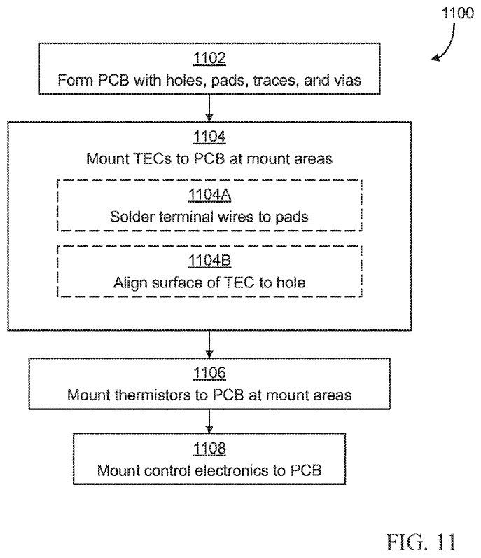

FIG. 11 illustrates an example of a process for forming a PCB having mount areas with TECs mounted thereto, such as the apparatus 800 of FIG. 8A. The process 1100 begins in block 1102, in which a PCB is formed having traces, vias, and mount pads, in addition to holes. Any suitable technique, including known techniques, may be used to form the traces, vias, mount pads, and layers of the PCB. The holes of the mount areas may be formed in any suitable manner, including by forming the PCB with no material in the areas corresponding to the holes or by forming the PCB with material in the holes and etching, cutting, or otherwise excising the material from the holes. The actions of block 1102 may be performed by conventional PCB manufacturing equipment or any other suitable PCB manufacturing equipment.

In block 1104, TECs are attached to the PCB at the mount areas, with one TEC attached at each mount area. The TECs may be attached to the PCB using circuit manufacturing or assembly equipment, including surface mount technology (SMT) assembly equipment such as pick-and-place robotics equipment. As part of attaching TECs to the mount areas, terminal wires of the TEC are soldered to mount pads of the mount area (1104A). In some embodiments, the soldering may be performed by circuit manufacture equipment including selective soldering equipment or other soldering equipment. When the terminal wires are soldered to the mount pads, the TEC may be aligned with the hole of the mount area such that a surface of the TEC to be heated or cooled is aligned with the hole (1104B). When the surface of the TEC is aligned with the hole, the surface may be positioned such that the whole surface is disposed within an area defined by the edges of the hole. When aligned, the surface of the TEC may be three-dimensionally positioned above the hole, or may be positioned such that the surface of the TEC (and some or all of the TEC apart from the surface) is within the hole.

In block 1106, the thermistor is mounted to the mount area, at a side of the PCB opposite the side on which the TEC is mounted. The thermistor may be mounted using circuit manufacture equipment, including surface mount technology (SMT) assembly equipment such as pick-and-place robotics equipment. To mount the thermistor to the mount area, the thermistor may be soldered to the mount area, and selective soldering equipment or other soldering equipment may be used.

In block 1108, in addition to the TECs and thermistors being mounted on the PCB, control electronics may also be mounted to the PCB at a control circuit mount area. The control electronics may be arranged in any suitable manner, including as an integrated circuit. Depending on the form of the control electronics, different mounting styles or mounting equipment may be used. In some embodiments in which the control electronics are formed as one or more integrated circuits, the integrated circuits may be mounted using circuit manufacture equipment, including surface mount technology (SMT) assembly equipment such as pick-and-place robotics equipment. To mount the control electronics to the mount area, the integrated circuit may be soldered to the mount area, and selective soldering equipment or other soldering equipment may be used.

Once the components are mounted to the PCB, the process 1100 ends.

### Amplification (AMP) Methods

Described herein are methods of determining the nucleotide sequence contiguous to a known target nucleotide sequence. The methods may be implemented in an automated fashion using the systems disclosed herein. Traditional sequencing methods generate sequence information randomly (e.g., “shotgun” sequencing) or between two known sequences which are used to design primers. In contrast, certain of the methods described herein, in some embodiments, allow for determining the nucleotide sequence (e.g., sequencing) upstream or downstream of a single region of known sequence with a high level of specificity and sensitivity.

In some embodiments, the systems provided herein may be configured to implement, e.g., in an automated fashion, a method of enriching specific nucleotide sequences prior to determining the nucleotide sequence using a next-generation sequencing technology. In some embodiments, methods provided herein can relate to enriching samples comprising deoxyribonucleic acid (DNA). In some embodiments, methods provided herein comprise: (a) ligating a target nucleic acid comprising the known target nucleotide sequence with a universal oligonucleotide tail-adapter; (b) amplifying a portion of the target nucleic acid and the amplification strand of the universal oligonucleotide tail-adapter with a first adapter primer and a first target-specific primer; (c) amplifying a portion of the amplicon resulting from step (b) with a second adapter primer and a second target-specific primer; and (d) transferring the DNA solution to a user. In some embodiments, one or more steps of the methods may be performed within different vessels of a cartridge provided herein. In some embodiments, microfluidic channels and valves in the cartridge facilitate the transfer of reaction material/fluid from one vessel to another in the cartridge to permit reactions to proceed in an automated fashion. In some embodiments, a DNA solution can subsequently be sequenced with a first and second sequencing primer using a next-generation sequencing technology.

In some embodiments, a sample processed using a system provided herein comprises genomic DNA. In some embodiments, samples comprising genomic DNA include a fragmentation step preceding step (a). In some embodiments, each ligation and amplification step can optionally comprise a subsequent purification step (e.g., sample purification between step (a) and step (b), sample purification between step (b) and step (c), and/or sample purification following step (c)). For example, the method of enriching samples comprising genomic DNA can comprise: (a) fragmentation of genomic DNA; (b) ligating a target nucleic acid comprising the known target nucleotide sequence with a universal oligonucleotide tail-adapter; (c) post-ligation sample purification; (d) amplifying a portion of the target nucleic acid and the amplification strand of the universal oligonucleotide tail-adapter with a first adapter primer and a first target-specific primer; (e) post-amplification sample purification; (f) amplifying a portion of the amplicon resulting from step (d) with a second adapter primer and a second target-specific primer; (g) post-amplification sample purification; and (h) transferring the purified DNA solution to a user. In some embodiments, steps of the methods may be performed within different vessels of a cartridge provided herein. In some embodiments, microfluidic channels and valves in the cartridge facilitate the transfer of reaction material/fluid from one vessel to another in the cartridge in an automated fashion. In The purified sample can subsequently be sequenced with a first and second sequencing primer using a next-generation sequencing technology.

In some embodiments, systems and methods provided herein may be used for processing nucleic acids as depicted in the exemplary workflow in FIG. 1. A nucleic acid sample 120 is provided. In some embodiments, the sample comprises RNA. In some embodiments, the sample comprises DNA (e.g., double-stranded complementary DNA (cDNA) and/or double-stranded genomic DNA (gDNA) 102). In some embodiments, the nucleic acid sample is subjected to a step 102 comprising nucleic acid end repair and/or dA tailing. In some embodiments, the nucleic acid sample is subjected to a step 104 comprising adapter ligation. In some embodiments, a universal oligonucleotide adapter 122 is ligated to one or more nucleic acids in the nucleic acid sample. In some embodiments, the ligation step comprises blunt-end ligation. In some embodiments, the ligation step comprises sticky-end ligation. In some embodiments, the ligation step comprises overhang ligation. In some embodiments, the ligation step comprises TA ligation. In some embodiments, the dA tailing step 102 is performed to generate an overhang in the nucleic acid sample that is complementary to an overhang in the universal oligonucleotide adapter (e.g., TA ligation). In some embodiments, a universal oligonucleotide adapter is ligated to both ends of one or more nucleic acids in the nucleic acid sample to generate a nucleic acid 124 flanked by universal oligonucleotide adapters. In some embodiments, an initial round of amplification is performed using an adapter primer 130 and a first target-specific primer 132. In some embodiments, the amplified sample is subjected to a second round of amplification using an adapter primer and a second target-specific primer 134. In some embodiments, the second target-specific primer is nested relative to the first target-specific primer. In some embodiments, the second target-specific primer comprises additional sequences 5′ to a hybridization sequence (e.g., common sequence) that may include barcode, index, adapter sequences, or sequencing primer sites. In some embodiments, the second target-specific primer is further contacted by an additional primer that hybridizes with the common sequence of the second target-specific primer, as depicted by 134. In some embodiments, the second round of amplification generates a nucleic acid 126 that is suitable for nucleic acid sequencing (e.g., next generation sequencing methods).

In some embodiments, systems and methods provided herein may be used for processing nucleic acids as described in PCT International Application No. PCT/US2017/051924, which was filed on Sep. 15, 2017, and which claims priority under 35 U.S.C. § 119(e) to U.S. Provisional Patent Application No. 62/395,339, which was filed on Sep. 15, 2016, and in PCT International Application No. PCT/US2017/051927, which was filed on Sep. 15, 2017, and which claims priority under 35 U.S.C. § 119(e) to U.S. Provisional Patent Application No. 62/395,347, which was filed on Sep. 15, 2016, the entire contents of each of which relating to nucleic acid library preparation are hereby incorporated by reference.

In some embodiments, a sample processed using a system provided herein comprises ribonucleic acid (RNA). In some embodiments, a system provided herein can be useful for processing RNA by a method comprising: (a) contacting a target nucleic acid molecule comprising the known target nucleotide sequence with a population of random primers under hybridization conditions; (b) performing a template-dependent extension reaction that is primed by a hybridized random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) subjecting the nucleic acid to end-repair, phosphorylation, and adenylation; (f) ligating the target nucleic acid comprising the known target nucleotide sequence with a universal oligonucleotide tail-adapter; (g) amplifying a portion of the target nucleic acid and the amplification strand of the universal oligonucleotide tail-adapter with a first adapter primer and a first target-specific primer; (h) amplifying a portion of the amplicon resulting from step (g) with a second adapter primer and a second target-specific primer; and (i) transferring the cDNA solution to a user. In some embodiments, one or more steps of the methods may be performed within different vessels of a cartridge provided herein. In some embodiments, cDNA solution can subsequently be sequenced with a first and second sequencing primer using a next-generation sequencing technology.

In some embodiments, each ligation and amplification step can optionally comprise a subsequent sample purification step (e.g., sample purification step between step (f) and step (g), sample purification step between step (g) and step (h), and/or sample purification following step (h)). For example, the method of enriching samples comprising RNA can comprise: (a) contacting a target nucleic acid molecule comprising the known target nucleotide sequence with a population of random primers under hybridization conditions; (b) performing a template-dependent extension reaction that is primed by a hybridized random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) subjecting the nucleic acid to end-repair, phosphorylation, and adenylation; (f) ligating the target nucleic acid comprising the known target nucleotide sequence with a universal oligonucleotide tail-adapter; (g) post-ligation sample purification; (h) amplifying a portion of the target nucleic acid and the amplification strand of the universal oligonucleotide tail-adapter with a first adapter primer and a first target-specific primer; (i) post-amplification sample purification; (j) amplifying a portion of the amplicon resulting from step (h) with a second adapter primer and a second target-specific primer; (k) post-amplification sample purification; and (l) transferring the purified cDNA solution to a user. In some embodiments, one or more steps of the methods may be performed within different vessels of a cartridge provided herein. The purified sample can subsequently be sequenced with a first and second sequencing primer using a next-generation sequencing technology.

In some embodiments, the systems provided herein may be configured to implement, e.g., in an automated fashion, a method of enriching nucleotide sequences that comprise a known target nucleotide sequence downstream from an adjacent region of unknown nucleotide sequence (e.g., nucleotide sequences comprising a 5′ region comprising an unknown sequence and a 3′ region comprising a known sequence). In some embodiments, the method comprises: (a) contacting a target nucleic acid molecule comprising the known target nucleotide sequence with an initial target-specific primer under hybridization conditions; (b) performing a template-dependent extension reaction that is primed by a hybridized initial target-specific primer and that uses the target nucleic acid molecule as a template; (c) contacting the product of step (b) with a population of tailed random primers under hybridization conditions; (d) performing a template-dependent extension reaction that is primed by a hybridized tailed random primer and that uses the portion of the target nucleic acid molecule downstream of the site of hybridization as a template; (e) amplifying a portion of the target nucleic acid molecule and the tailed random primer sequence with a first tail primer and a first target-specific primer; (f) amplifying a portion of the amplicon resulting from step (e) with a second tail primer and a second target-specific primer; and (g) transferring the cDNA solution to a user. The cDNA solution can subsequently be sequenced with a first and second sequencing primer using a next-generation sequencing technology. In some embodiments, the population of tailed random primers comprises single-stranded oligonucleotide molecules having a 5′ nucleic acid sequence identical to a first sequencing primer and a 3′ nucleic acid sequence comprising from about 6 to about 12 random nucleotides. In some embodiments, the first target-specific primer comprises a nucleic acid sequence that can specifically anneal to the known target nucleotide sequence of the target nucleic acid at the annealing temperature. In some embodiments, the second target-specific primer comprises a 3′ portion comprising a nucleic acid sequence that can specifically anneal to a portion of the known target nucleotide sequence comprised by the amplicon resulting from step (e), and a 5′ portion comprising a nucleic acid sequence that is identical to a second sequencing primer and the second target-specific primer is nested with respect to the first target-specific primer. In some embodiments, the first tail primer comprises a nucleic acid sequence identical to the tailed random primer. In some embodiments, the second tail primer comprises a nucleic acid sequence identical to a portion of the first sequencing primer and is nested with respect to the first tail primer. In some embodiments, one or more steps of the method may be performed within different vessels of a cartridge provided herein.

In some embodiments, the systems provided herein may be configured to implement, e.g., in an automated fashion, a method of enriching nucleotide sequences that comprise a known target nucleotide sequence upstream from an adjacent region of unknown nucleotide sequence (e.g., nucleotide sequences comprising a 5′ region comprising a known sequence and a 3′ region comprising an unknown sequence). In some embodiments, the method comprises: (a) contacting a target nucleic acid molecule comprising the known target nucleotide sequence with a population of tailed random primers under hybridization conditions; (b) performing a template-dependent extension reaction that is primed by a hybridized tailed random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) amplifying a portion of the target nucleic acid molecule and the tailed random primer sequence with a first tail primer and a first target-specific primer; (f) amplifying a portion of the amplicon resulting from step (e) with a second tail primer and a second target-specific primer; and (g) transferring the cDNA solution to a user. The cDNA solution can subsequently be sequenced with a first and second sequencing primer using a next-generation sequencing technology. In some embodiments, the population of tailed random primers comprises single-stranded oligonucleotide molecules having a 5′ nucleic acid sequence identical to a first sequencing primer and a 3′ nucleic acid sequence comprising from about 6 to about 12 random nucleotides. In some embodiments, the first target-specific primer comprises a nucleic acid sequence that can specifically anneal to the known target nucleotide sequence of the target nucleic acid at the annealing temperature. In some embodiments, the second target-specific primer comprises a 3′ portion comprising a nucleic acid sequence that can specifically anneal to a portion of the known target nucleotide sequence comprised by the amplicon resulting from step (c), and a 5′ portion comprising a nucleic acid sequence that is identical to a second sequencing primer and the second target-specific primer is nested with respect to the first target-specific primer. In some embodiments, the first tail primer comprises a nucleic acid sequence identical to the tailed random primer. In some embodiments, the second tail primer comprises a nucleic acid sequence identical to a portion of the first sequencing primer and is nested with respect to the first tail primer. In some embodiments, one or more steps of the method may be performed within different vessels of a cartridge provided herein. In some embodiments, the method further involves a step of contacting the sample with RNase after extension of the initial target-specific primer. In some embodiments, the tailed random primer can form a hair-pin loop structure. In some embodiments, the initial target-specific primer and the first target-specific primer are identical. In some embodiments, the tailed random primer further comprises a barcode portion comprising 6-12 random nucleotides between the 5′ nucleic acid sequence identical to a first sequencing primer and the 3′ nucleic acid sequence comprising 6-12 random nucleotides.

### Universal Oligonucleotide Tail Adapter

As used herein, the term “universal oligonucleotide tail-adapter” refers to a nucleic acid molecule comprised of two strands (a blocking strand and an amplification strand) and comprising a first ligatable duplex end and a second unpaired end. The blocking strand of the universal oligonucleotide tail-adapter comprises a 5′ duplex portion. The amplification strand comprises an unpaired 5′ portion, a 3′ duplex portion, a 3′ T overhang, and nucleic acid sequences identical to a first and second sequencing primer. The duplex portions of the blocking strand and the amplification strand are substantially complementary and form the first ligatable duplex end comprising a 3′ T overhang and the duplex portion is of sufficient length to remain in duplex form at the ligation temperature.

In some embodiments, the portion of the amplification strand that comprises a nucleic acid sequence identical to a first and second sequencing primer can be comprised, at least in part, by the 5′ unpaired portion of the amplification strand.

In some embodiments, the universal oligonucleotide tail-adapter can comprise a duplex portion and an unpaired portion, wherein the unpaired portion comprises only the 5′ portion of the amplification strand, i.e., the entirety of the blocking strand is a duplex portion.

In some embodiments, the universal oligonucleotide tail-adapter can have a “Y” shape, i.e., the unpaired portion can comprise portions of both the blocking strand and the amplification strand which are unpaired. The unpaired portion of the blocking strand can be shorter than, longer than, or equal in length to the unpaired portion of the amplification strand. In some embodiments, the unpaired portion of the blocking strand can be shorter than the unpaired portion of the amplification strand. Y shaped universal oligonucleotide tail-adapters have the advantage that the unpaired portion of the blocking strand will not be subject to 3′ extension during a PCR regimen.

In some embodiments, the blocking strand of the universal oligonucleotide tail-adapter can further comprise a 3′ unpaired portion which is not substantially complementary to the 5′ unpaired portion of the amplification strand; and wherein the 3′ unpaired portion of the blocking strand is not substantially complementary to or substantially identical to any of the primers. In some embodiments, the blocking strand of the universal oligonucleotide tail-adapter can further comprise a 3′ unpaired portion which will not specifically anneal to the 5′ unpaired portion of the amplification strand at the annealing temperature; and wherein the 3′ unpaired portion of the blocking strand will not specifically anneal to any of the primers or the complements thereof at the annealing temperature.

### First Amplification Step

As used herein, the term “first target-specific primer” refers to a single-stranded oligonucleotide comprising a nucleic acid sequence that can specifically anneal under suitable annealing conditions to a nucleic acid template that has a strand characteristic of a target nucleic acid.

In some embodiments, a primer (e.g., a target specific primer) can comprise a 5′ tag sequence portion. In some embodiments, multiple primers (e.g., all first-target specific primers) present in a reaction can comprise identical 5′ tag sequence portions. In some embodiments, in a multiplex PCR reaction, different primer species can interact with each other in an off-target manner, leading to primer extension and subsequently amplification by DNA polymerase. In such embodiments, these primer dimers tend to be short, and their efficient amplification can overtake the reaction and dominate resulting in poor amplification of desired target sequence. Accordingly, in some embodiments, the inclusion of a 5′ tag sequence in primers (e.g., on target specific primer(s)) may result in formation of primer dimers that contain the same complementary tails on both ends. In some embodiments, in subsequent amplification cycles, such primer dimers would denature into single-stranded DNA primer dimers, each comprising complementary sequences on their two ends which are introduced by the 5′ tag. In some embodiments, instead of primer annealing to these single stranded DNA primer dimers, an intra-molecular hairpin (a panhandle like structure) formation may occur due to the proximate accessibility of the complementary tags on the same primer dimer molecule instead of an inter-molecular interaction with new primers on separate molecules. Accordingly, in some embodiments, these primer dimers may be inefficiently amplified, such that primers are not exponentially consumed by the dimers for amplification; rather the tagged primers can remain in high and sufficient concentration for desired specific amplification of target sequences. In some embodiments, accumulation of primer dimers may be undesirable in the context of multiplex amplification because they compete for and consume other reagents in the reaction.

In some embodiments, a 5′ tag sequence can be a GC-rich sequence. In some embodiments, a 5′ tag sequence may comprise at least 50% GC content, at least 55% GC content, at least 60% GC content, at least 65% GC content, at least 70% GC content, at least 75% GC content, at least 80% GC content, or higher GC content. In some embodiments, a tag sequence may comprise at least 60% GC content. In some embodiments, a tag sequence may comprise at least 65% GC content.

As used herein, the term “first adapter primer” refers to a nucleic acid molecule comprising a nucleic acid sequence identical to a 5′ portion of the first sequencing primer. As the first tail-adapter primer is therefore identical to at least a portion of the sequence of the amplification strand (as opposed to complementary), it will not be able to specifically anneal to any portion of the universal oligonucleotide tail-adapter itself.

In the first PCR amplification cycle of the first amplification step, the first target-specific primer can specifically anneal to a template strand of any nucleic acid comprising the known target nucleotide sequence. Depending upon the orientation with which the first target-specific primer was designed, a sequence upstream or downstream of the known target nucleotide sequence will be synthesized as a strand complementary to the template strand. If, during the extension phase of PCR, the 5′ end of the template strand terminates in a ligated universal oligonucleotide tail-adapter, the 3′ end of the newly synthesized product strand will comprise sequence complementary to the first tail-adapter primer. In subsequent PCR amplification cycles, both the first target-specific primer and the first tail-adapter primer will be able to specifically anneal to the appropriate strands of the target nucleic acid sequence and the sequence between the known nucleotide target sequence and the universal oligonucleotide tail-adapter can be amplified (i.e., copied).

### Second Amplification Step

As used herein, the term “second target-specific primer” refers to a single-stranded oligonucleotide comprising a 3′ portion comprising a nucleic acid sequence that can specifically anneal to a portion of the known target nucleotide sequence comprised by the amplicon resulting from a preceding amplification step, and a 5′ portion comprising a nucleic acid sequence that is identical to a second sequencing primer. The second target-specific primer can be further contacted by an additional primer (e.g., a primer having 3′ sequencing adapter/index sequences) that hybridizes with the common sequence of the second target-specific primer. In some embodiments, the additional primer may comprise additional sequences 5′ to the hybridization sequence that may include barcode, index, adapter sequences, or sequencing primer sites. In some embodiments, the additional primer is a generic sequencing adapter/index primer. The second target-specific primer is nested with respect to the first target-specific primer. In some embodiments, the second target-specific primer is nested with respect to the first target-specific primer by at least 3 nucleotides, e.g., by 3 or more, 4 or more, 5 or more, 6 or more, 7 or more, 8 or more, 9 or more, 10 or more, or 15 or more nucleotides.

In some embodiments, all of the second target-specific primers present in a reaction comprise the same 5′ portion. In some embodiments, the 5′ portion of the second target-specific primers can serve to suppress primer dimers as described for the 5′ tag of the first target-specific primer described above herein.

In some embodiments, the first and second target-specific primers are substantially complementary to the same strand of the target nucleic acid. In some embodiments, the portions of the first and second target-specific primers that specifically anneal to the known target sequence can comprise a total of at least 20 unique bases of the known target nucleotide sequence, e.g., 20 or more unique bases, 25 or more unique bases, 30 or more unique bases, 35 or more unique bases, 40 or more unique bases, or 50 or more unique bases. In some embodiments, the portions of the first and second target-specific primers that specifically anneal to the known target sequence can comprise a total of at least 30 unique bases of the known target nucleotide sequence.

As used herein, the term “second adapter primer” refers to a nucleic acid molecule comprising a nucleic acid sequence identical to a portion of the first sequencing primer and is nested with respect to the first adapter primer. As the second tail-adapter primer is therefore identical to at least a portion of the sequence of the amplification strand (as opposed to complementary), it will not be able to specifically anneal to any portion of the universal oligonucleotide tail-adapter itself. In some embodiments, the second adapter primer is identical to the first sequencing primer.

The second adapter primer should be nested with respect to the first adapter primer, that is, the first adapter primer comprises a nucleic acid sequence identical to the amplification strand which is not comprised by the second adapter primer and which is located closer to the 5′ end of the amplification primer than any of the sequence identical to the amplification strand which is comprised by the second adapter primer. In some embodiments, the second adapter primer is nested by at least 3 nucleotides, e.g., by 3 nucleotides, by 4 nucleotides, by 5 nucleotides, by 6 nucleotides, by 7 nucleotides, by 8 nucleotides, by 9 nucleotides, by 10 nucleotides or more.

In some embodiments, the first adapter primer can comprise a nucleic acid sequence identical to about the 205′-most bases of the amplification strand of the universal oligonucleotide tail-adapter and the second adapter primer can comprise a nucleic acid sequence identical to about 30 bases of the amplification strand of the universal oligonucleotide tail-adapter, with a 5′ base which is at least 3 nucleotides 3′ of the 5′ terminus of the amplification strand.

In some embodiments, nested primer sets may be used. In some embodiments, the use of nested adapter primers eliminates the possibility of producing final amplicons that are amplifiable (e.g., during bridge PCR or emulsion PCR) but cannot be efficiently sequenced using certain techniques. In some embodiments, hemi-nested primer sets may be used.

### Sample Purification Step

In some embodiments, target nucleic acids and/or amplification products thereof can be isolated from enzymes, primers, or buffer components before and/or after any appropriate step of a method. Any suitable methods for isolating nucleic acids may be used. In some embodiments, the isolation can comprise Solid Phase Reversible Immobilization (SPRI) cleanup. Methods for SPRI cleanup are well known in the art, e.g., Agencourt AMPure XP-PCR Purification (Cat No. A63880, Beckman Coulter; Brea, Calif.). In some embodiments, enzymes can be inactivated by heat treatment.

In some embodiments, unhybridized primers can be removed from a nucleic acid preparation using appropriate methods (e.g., purification, digestion, etc.). In some embodiments, a nuclease (e.g., exonuclease I) is used to remove primer from a preparation. In some embodiments, such nucleases are heat inactivated subsequent to primer digestion. Once the nucleases are inactivated, a further set of primers may be added together with other appropriate components (e.g., enzymes, buffers) to perform a further amplification reaction.

### Sequencing

In some aspects, the technology described herein relates to methods of enriching nucleic acid samples for oligonucleotide sequencing. In some embodiments, the sequencing can be performed by a next-generation sequencing method. As used herein, “next-generation sequencing” refers to oligonucleotide sequencing technologies that have the capacity to sequence oligonucleotides at speeds above those possible with conventional sequencing methods (e.g., Sanger sequencing), due to performing and reading out thousands to millions of sequencing reactions in parallel. Non-limiting examples of next-generation sequencing methods/platforms include Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ION Torrent); DNA nanoball sequencing (Complete Genomics); and technologies available from Pacific Biosciences, Intelligen Bio-systems, and Oxford Nanopore Technologies. In some embodiments, the sequencing primers can comprise portions compatible with the selected next-generation sequencing method. Next-generation sequencing technologies and the constraints and design parameters of associated sequencing primers are well known in the art (see, e.g., Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; Zhang et al., “The impact of next-generation sequencing on genomics”, J Genet Genomics, 2011, 38(3):95-109; (Nyren, P. et al. Anal Biochem 208: 17175 (1993); Bentley, D. R. Curr Opin Genet Dev 16:545-52 (2006); Strausberg, R. L., et al. Drug Disc Today 13:569-77 (2008); U.S. Pat. Nos. 7,282,337; 7,279,563; 7,226,720; 7,220,549; 7,169,560; 6,818,395; 6,911,345; US Pub. Nos. 2006/0252077; 2007/0070349; and 20070070349; which are incorporated by reference herein in their entireties).

In some embodiments, the sequencing step relies upon the use of a first and second sequencing primer. In some embodiments, the first and second sequencing primers are selected to be compatible with a next-generation sequencing method as described herein.

Methods of aligning sequencing reads to known sequence databases of genomic and/or cDNA sequences are well known in the art, and software is commercially available for this process. In some embodiments, reads (less the sequencing primer and/or adapter nucleotide sequence) which do not map, in their entirety, to wild-type sequence databases can be genomic rearrangements or large indel mutations. In some embodiments, reads (less the sequencing primer and/or adapter nucleotide sequence) comprising sequences which map to multiple locations in the genome can be genomic rearrangements.

### AMP Primers

In some embodiments, the four types of primers (first and second target-specific primers and first and second adapter primers) are designed such that they will specifically anneal to their complementary sequences at an annealing temperature of from about 61 to 72° C., e.g., from about 61 to 69° C., from about 63 to 69° C., from about 63 to 67° C., from about 64 to 66° C. In some embodiments, the four types of primers are designed such that they will specifically anneal to their complementary sequences at an annealing temperature of less than 72° C. In some embodiments, the four types of primers are designed such that they will specifically anneal to their complementary sequences at an annealing temperature of less than 70° C. In some embodiments, the four types of primers are designed such that they will specifically anneal to their complementary sequences at an annealing temperature of less than 68° C. In some embodiments, the four types of primers are designed such that they will specifically anneal to their complementary sequences at an annealing temperature of about 65° C. In some embodiments, systems provided herein are configured to alter vessel temperature (e.g., by cycling between different temperature ranges) to facilitate primer annealing.

In some embodiments, the portions of the target-specific primers that specifically anneal to the known target nucleotide sequence will anneal specifically at a temperature of about 61 to 72° C., e.g., from about 61 to 69° C., from about 63 to 69° C., from about 63 to 67° C., from about 64 to 66° C. In some embodiments, the portions of the target-specific primers that specifically anneal to the known target nucleotide sequence will anneal specifically at a temperature of about 65° C. in a PCR buffer.

In some embodiments, the primers and/or adapters described herein cannot comprise modified bases (e.g., the primers and/or adapters cannot comprise a blocking 3′ amine).

### Nucleic Acid Extension, Amplification, and PCR

In some embodiments, methods described herein comprise an extension regimen or step. In such embodiments, extension may proceed from one or more hybridized tailed random primers, using the nucleic acid molecules which the primers are hybridized to as templates. Extension steps are described herein. In some embodiments, one or more tailed random primers can hybridize to substantially all of the nucleic acids in a sample, many of which may not comprise a known target nucleotide sequence. Accordingly, in some embodiments, extension of random primers may occur due to hybridization with templates that do not comprise a known target nucleotide sequence.

In some embodiments, methods described herein may involve a polymerase chain reaction (PCR) amplification regimen, involving one or more amplification cycles. Amplification steps of the methods described herein can each comprise a PCR amplification regimen, i.e., a set of polymerase chain reaction (PCR) amplification cycles. In some embodiments, systems provided herein are configured to alter vessel temperature (e.g., by cycling between different temperature ranges) to facilitate different PCR steps, e.g., melting, annealing, elongation, etc.

In some embodiments, system provided herein are configured to implement an amplification regimen in an automated fashion. As used herein, the term “amplification regimen” refers to a process of specifically amplifying (increasing the abundance of) a nucleic acid of interest. In some embodiments, exponential amplification occurs when products of a previous polymerase extension serve as templates for successive rounds of extension. In some embodiments, a PCR amplification regimen according to methods disclosed herein may comprise at least one, and in some cases at least 5 or more iterative cycles. In some embodiments, each iterative cycle comprises steps of: 1) strand separation (e.g., thermal denaturation); 2) oligonucleotide primer annealing to template molecules; and 3) nucleic acid polymerase extension of the annealed primers. In should be appreciated that any suitable conditions and times involved in each of these steps may be used. In some embodiments, conditions and times selected may depend on the length, sequence content, melting temperature, secondary structural features, or other factors relating to the nucleic acid template and/or primers used in the reaction. In some embodiments, an amplification regimen according to methods described herein is performed in a thermal cycler, many of which are commercially available.

In some embodiments, a nucleic acid extension reaction involves the use of a nucleic acid polymerase. As used herein, the phrase “nucleic acid polymerase” refers an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and are commercially available. One group of nucleic acid polymerases are thermostable, i.e., they retain function after being subjected to temperatures sufficient to denature annealed strands of complementary nucleic acids, e.g., 94° C., or sometimes higher. A non-limiting example of a protocol for amplification involves using a polymerase (e.g., Phoenix Taq, VeraSeq) under the following conditions: 98° C. for 30 s, followed by 14-22 cycles comprising melting at 98° C. for 10 s, followed by annealing at 68° C. for 30 s, followed by extension at 72° C. for 3 min, followed by holding of the reaction at 4° C. However, other appropriate reaction conditions may be used. In some embodiments, annealing/extension temperatures may be adjusted to account for differences in salt concentration (e.g., 3° C. higher to higher salt concentrations). In some embodiments, slowing the ramp rate (e.g., 1° C./s, 0.5° C./s, 0.28° C./s, 0.1° C./s or slower), for example, from 98° C. to 65° C., improves primer performance and coverage uniformity in highly multiplexed samples. In some embodiments, systems provided herein are configured to alter vessel temperature (e.g., by cycling between different temperature ranges, having controlled ramp up or down rates) to facilitate amplification.

In some embodiments, a nucleic acid polymerase is used under conditions in which the enzyme performs a template-dependent extension. In some embodiments, the nucleic acid polymerase is DNA polymerase I, Taq polymerase, Phoenix Taq polymerase, Phusion polymerase, T4 polymerase, T7 polymerase, Klenow fragment, Klenow exo-, phi29 polymerase, AMV reverse transcriptase, M-MuLV reverse transcriptase, HIV-1 reverse transcriptase, VeraSeq ULtra polymerase, VeraSeq HF 2.0 polymerase, EnzScript, or another appropriate polymerase. In some embodiments, a nucleic acid polymerase is not a reverse transcriptase. In some embodiments, a nucleic acid polymerase acts on a DNA template. In some embodiments, the nucleic acid polymerase acts on an RNA template. In some embodiments, an extension reaction involves reverse transcription performed on an RNA to produce a complementary DNA molecule (RNA-dependent DNA polymerase activity). In some embodiments, a reverse transcriptase is a mouse moloney murine leukemia virus (M-MLV) polymerase, AMV reverse transcriptase, RSV reverse transcriptase, HIV-1 reverse transcriptase, HIV-2 reverse transcriptase, or another appropriate reverse transcriptase.

In some embodiments, a nucleic acid amplification reaction involves cycles including a strand separation step generally involving heating of the reaction mixture. As used herein, the term “strand separation” or “separating the strands” means treatment of a nucleic acid sample such that complementary double-stranded molecules are separated into two single strands available for annealing to an oligonucleotide primer. In some embodiments, strand separation according to methods described herein is achieved by heating the nucleic acid sample above its melting temperature (Tₘ). In some embodiments, for a sample containing nucleic acid molecules in a reaction preparation suitable for a nucleic acid polymerase, heating to 94° C. is sufficient to achieve strand separation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), and a carrier (e.g., 0.01 to 0.5% BSA). A non-limiting example of a suitable buffer comprises 50 mM KCl, 10 mM Tris-HCl (pH 8.8 at 25° C.), 0.5 to 3 mM MgCl₂, and 0.1% BSA.

In some embodiments, a nucleic acid amplification involves annealing primers to nucleic acid templates having a strands characteristic of a target nucleic acid. In some embodiments, a strand of a target nucleic acid can serve as a template nucleic acid.

As used herein, the term “anneal” refers to the formation of one or more complementary base pairs between two nucleic acids. In some embodiments, annealing involves two complementary or substantially complementary nucleic acid strands hybridizing together. In some embodiments, in the context of an extension reaction, annealing involves the hybridization of primer to a template such that a primer extension substrate for a template-dependent polymerase enzyme is formed. In some embodiments, conditions for annealing (e.g., between a primer and nucleic acid template) may vary based of the length and sequence of a primer. In some embodiments, conditions for annealing are based upon a Tₘ (e.g., a calculated Tₘ) of a primer. In some embodiments, an annealing step of an extension regimen involves reducing the temperature following a strand separation step to a temperature based on the Tₘ (e.g., a calculated Tₘ) for a primer, for a time sufficient to permit such annealing. In some embodiments, a Tₘ can be determined using any of a number of algorithms (e.g., OLIGO™ (Molecular Biology Insights Inc. Colorado) primer design software and VENTRO NTI™ (Invitrogen, Inc. California) primer design software and programs available on the internet, including Primer3, Oligo Calculator, and NetPrimer (Premier Biosoft; Palo Alto, Calif.; and freely available on the world wide web (e.g., at premierbiosoft.com/netprimer/netprlaunch/Help/xnetprlaunch.html)). In some embodiments, the Tₘ of a primer can be calculated using the following formula, which is used by NetPrimer software and is described in more detail in Frieir, et al. PNAS 198683:9373-9377 which is incorporated by reference herein in its entirety.

Tₘ=ΔH/(ΔS+R*ln(C/4))+16.6 log([K⁺]/(1+0.7[K⁺]))−273.15

wherein: ΔH is enthalpy for helix formation; ΔS is entropy for helix formation; R is molar gas constant (1.987 cal/° C.*mol); C is the nucleic acid concentration; and [K⁺] is salt concentration. For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can, for example, temperatures more than 5° C. below the predicted Tₘ (e.g., 6° C. below, 8° C. below, 10° C. below or lower). In some embodiments, the closer an annealing temperature is to the Tₘ, the more specific is the annealing. In some embodiments, the time used for primer annealing during an extension reaction (e.g., within the context of a PCR amplification regimen) is determined based, at least in part, upon the volume of the reaction (e.g., with larger volumes involving longer times). In some embodiments, the time used for primer annealing during an extension reaction (e.g., within the context of a PCR amplification regimen) is determined based, at least in part, upon primer and template concentrations (e.g., with higher relative concentrations of primer to template involving less time than lower relative concentrations). In some embodiments, depending upon volume and relative primer/template concentration, primer annealing steps in an extension reaction (e.g., within the context of an amplification regimen) can be in the range of 1 second to 5 minutes, 10 seconds to 2 minutes, or 30 seconds to 2 minutes. As used herein, “substantially anneal” refers to an extent to which complementary base pairs form between two nucleic acids that, when used in the context of a PCR amplification regimen, is sufficient to produce a detectable level of a specifically amplified product.

As used herein, the term “polymerase extension” refers to template-dependent addition of at least one complementary nucleotide, by a nucleic acid polymerase, to the 3′ end of a primer that is annealed to a nucleic acid template. In some embodiments, polymerase extension adds more than one nucleotide, e.g., up to and including nucleotides corresponding to the full length of the template. In some embodiments, conditions for polymerase extension are based, at least in part, on the identity of the polymerase used. In some embodiments, the temperature used for polymerase extension is based upon the known activity properties of the enzyme. In some embodiments, in which annealing temperatures are below the optimal temperatures for the enzyme, it may be acceptable to use a lower extension temperature. In some embodiments, enzymes may retain at least partial activity below their optimal extension temperatures. In some embodiments, a polymerase extension (e.g., performed with thermostable polymerases such as Taq polymerase and variants thereof) is performed at 65° C. to 75° C. or 68° C. to 72° C. In some embodiments, methods provided herein involve polymerase extension of primers that are annealed to nucleic acid templates at each cycle of a PCR amplification regimen. In some embodiments, a polymerase extension is performed using a polymerase that has relatively strong strand displacement activity. In some embodiments, polymerases having strong strand displacement are useful for preparing nucleic acids for purposes of detecting fusions (e.g., 5′ fusions).

In some embodiments, primer extension is performed under conditions that permit the extension of annealed oligonucleotide primers. As used herein, the term “conditions that permit the extension of an annealed oligonucleotide such that extension products are generated” refers to the set of conditions (e.g., temperature, salt and co-factor concentrations, pH, and enzyme concentration) under which a nucleic acid polymerase catalyzes primer extension. In some embodiments, such conditions are based, at least in part, on the nucleic acid polymerase being used. In some embodiments, a polymerase may perform a primer extension reaction in a suitable reaction preparation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), a carrier (e.g., 0.01 to 0.5% BSA), and one or more NTPs (e.g, 10 to 200 μM of each of dATP, dTTP, dCTP, and dGTP). A non-limiting set of conditions is 50 mM KCl, 10 mM Tris-HCl (pH 8.8 at 25° C.), 0.5 to 3 mM MgCl₂, 200 μM each dNTP, and 0.1% BSA at 72° C., under which a polymerase (e.g., Taq polymerase) catalyzes primer extension. In some embodiments, conditions for initiation and extension may include the presence of one, two, three or four different deoxyribonucleoside triphosphates (e.g., selected from dATP, dTTP, dCTP, and dGTP) and a polymerization-inducing agent such as DNA polymerase or reverse transcriptase, in a suitable buffer. In some embodiments, a “buffer” may include solvents (e.g., aqueous solvents) plus appropriate cofactors and reagents which affect pH, ionic strength, etc.

In some embodiments, systems provided herein are configured to implement in an automated fashion multiple nucleic acid amplification cycles. In some embodiments, nucleic acid amplification involve up to 5, up to 10, up to 20, up to 30, up to 40 or more rounds (cycles) of amplification. In some embodiments, nucleic acid amplification may comprise a set of cycles of a PCR amplification regimen from 5 cycles to 20 cycles in length. In some embodiments, an amplification step may comprise a set of cycles of a PCR amplification regimen from 10 cycles to 20 cycles in length. In some embodiments, each amplification step can comprise a set of cycles of a PCR amplification regimen from 12 cycles to 16 cycles in length. In some embodiments, an annealing temperature can be less than 70° C. In some embodiments, an annealing temperature can be less than 72° C. In some embodiments, an annealing temperature can be about 65° C. In some embodiments, an annealing temperature can be from about 61 to about 72° C.

In various embodiments, methods and compositions described herein relate to performing a PCR amplification regimen with one or more of the types of primers described herein. As used herein, “primer” refers to an oligonucleotide capable of specifically annealing to a nucleic acid template and providing a 3′ end that serves as a substrate for a template-dependent polymerase to produce an extension product which is complementary to the template. In some embodiments, a primer is single-stranded, such that the primer and its complement can anneal to form two strands. Primers according to methods and compositions described herein may comprise a hybridization sequence (e.g., a sequence that anneals with a nucleic acid template) that is less than or equal to 300 nucleotides in length, e.g., less than or equal to 300, or 250, or 200, or 150, or 100, or 90, or 80, or 70, or 60, or 50, or 40, or 30 or fewer, or 20 or fewer, or 15 or fewer, but at least 6 nucleotides in length. In some embodiments, a hybridization sequence of a primer may be 6 to 50 nucleotides in length, 6 to 35 nucleotides in length, 6 to 20 nucleotides in length, 10 to 25 nucleotides in length.

Any suitable method may be used for synthesizing oligonucleotides and primers. In some embodiments, commercial sources offer oligonucleotide synthesis services suitable for providing primers for use in methods and compositions described herein (e.g., INVITROGEN™ Custom DNA Oligos (Life Technologies, Grand Island, N.Y.) or custom DNA Oligos from Integrated DNA Technologies (Coralville, Iowa)).

### DNA Shearing/Fragmentation

Nucleic acids used herein (e.g., prior to sequencing) can be sheared, e.g., mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a nucleic acid can be mechanically sheared by sonication. In some embodiments, systems provided here may have one or more vessels, e.g., within a cassette that is fitted within a cartridge, in which nucleic acids are sheared, e.g., mechanically or enzymatically.

In some embodiments, a target nucleic acid is not sheared or digested. In some embodiments, nucleic acid products of preparative steps (e.g., extension products, amplification products) are not sheared or enzymatically digested.

In some embodiments, when a target nucleic acid is RNA, the sample can be subjected to a reverse transcriptase regimen to generate a DNA template and the DNA template can then be sheared. In some embodiments, target RNA can be sheared before performing a reverse transcriptase regimen. In some embodiments, a sample comprising target RNA can be used in methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers.

### Target Nucleic Acid

As used herein, the term “target nucleic acid” refers to a nucleic acid molecule of interest (e.g., a nucleic acid to be analyzed). In some embodiments, a target nucleic acid comprises both a target nucleotide sequence (e.g., a known or predetermined nucleotide sequence) and an adjacent nucleotide sequence which is to be determined (which may be referred to as an unknown sequence). A target nucleic acid can be of any appropriate length. In some embodiments, a target nucleic acid is double-stranded. In some embodiments, the target nucleic acid is DNA. In some embodiments, the target nucleic acid is genomic or chromosomal DNA (gDNA). In some embodiments, the target nucleic acid can be complementary DNA (cDNA). In some embodiments, the target nucleic acid is single-stranded. In some embodiments, the target nucleic acid can be RNA (e.g., mRNA, rRNA, tRNA, long non-coding RNA, microRNA).

In some embodiments, the target nucleic acid can be comprised by genomic DNA. In some embodiments, the target nucleic acid can be comprised by ribonucleic acid (RNA), e.g., mRNA. In some embodiments, the target nucleic acid can be comprised by cDNA. Many of the sequencing methods suitable for use in the methods described herein provide sequencing runs with optimal read lengths of tens to hundreds of nucleotide bases (e.g., Ion Torrent technology can produce read lengths of 200-400 bp). Target nucleic acids comprised, for example, by genomic DNA or mRNA, can be comprised by nucleic acid molecules which are substantially longer than this optimal read length. In order for the amplified nucleic acid portion resulting from the second amplification step to be of a suitable length for use in a particular sequencing technology, the average distance between the known target nucleotide sequence and an end of the target nucleic acid to which the universal oligonucleotide tail-adapter can be ligated should be as close to the optimal read length of the selected technology as possible. For example, if the optimal read-length of a given sequencing technology is 200 bp, then the nucleic acid molecules amplified in accordance with the methods described herein should have an average length of about 400 bp or less. Target nucleic acids comprised by, e.g., genomic DNA or mRNA, can be sheared, e.g., mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a target nucleic acid comprised by genomic DNA can be mechanically sheared by sonication.

In some embodiments, when the target nucleic acid is comprised by RNA, the sample can be subjected to a reverse transcriptase regimen to generate a DNA template and the DNA template can then be sheared. In some embodiments, target RNA can be sheared before performing the reverse transcriptase regimen. In some embodiments, a sample comprising target RNA can be used in the methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers; and by subjecting the nucleic acid to end-repair, phosphorylation, and adenylation.

In some embodiments, the known target nucleotide sequence can be comprised by a gene rearrangement. The methods described herein are suited for determining the presence and/or identity of a gene rearrangement as the identity of only one half of the gene rearrangement must be previously known (i.e., the half of the gene rearrangement which is to be targeted by the gene-specific primers). In some embodiments, the gene rearrangement can comprise an oncogene. In some embodiments, the gene rearrangement can comprise a fusion oncogene.

As used herein, the term “known target nucleotide sequence” refers to a portion of a target nucleic acid for which the sequence (e.g., the identity and order of the nucleotide bases of the nucleic acid) is known. For example, in some embodiments, a known target nucleotide sequence is a nucleotide sequence of a nucleic acid that is known or that has been determined in advance of an interrogation of an adjacent unknown sequence of the nucleic acid. A known target nucleotide sequence can be of any appropriate length.

In some embodiments, a target nucleotide sequence (e.g., a known target nucleotide sequence) has a length of 10 or more nucleotides, 30 or more nucleotides, 40 or more nucleotides, 50 or more nucleotides, 100 or more nucleotides, 200 or more nucleotides, 300 or more nucleotides, 400 or more nucleotides, 500 or more nucleotides. In some embodiments, a target nucleotide sequence (e.g., a known target nucleotide sequence) has a length in the range of 10 to 100 nucleotides, 10 to 500 nucleotides, 10 to 1000 nucleotides, 100 to 500 nucleotides, 100 to 1000 nucleotides, 500 to 1000 nucleotides, 500 to 5000 nucleotides.

In some embodiments, methods are provided herein for determining sequences of contiguous (or adjacent) portions of a nucleic acid. As used herein, the term “nucleotide sequence contiguous to” refers to a nucleotide sequence of a nucleic acid molecule (e.g., a target nucleic acid) that is immediately upstream or downstream of another nucleotide sequence (e.g., a known nucleotide sequence). In some embodiments, a nucleotide sequence contiguous to a known target nucleotide sequence may be of any appropriate length. In some embodiments, a nucleotide sequence contiguous to a known target nucleotide sequence comprises 1 kb or less of nucleotide sequence, e.g., 1 kb or less of nucleotide sequence, 750 bp or less of nucleotide sequence, 500 bp or less of nucleotide sequence, 400 bp or less of nucleotide sequence, 300 bp or less of nucleotide sequence, 200 bp or less of nucleotide sequence, 100 bp or less of nucleotide sequence. In some embodiments, in which a sample comprises different target nucleic acids comprising a known target nucleotide sequence (e.g., a cell in which a known target nucleotide sequence occurs multiple times in its genome, or on separate, non-identical chromosomes), there may be multiple sequences which comprise “a nucleotide sequence contiguous to” the known target nucleotide sequence. As used herein, the term “determining a (or the) nucleotide sequence,” refers to determining the identity and relative positions of the nucleotide bases of a nucleic acid.

In some embodiments, a known target nucleic acid can contain a fusion sequence resulting from a gene rearrangement. In some embodiments, methods described herein are suited for determining the presence and/or identity of a gene rearrangement. In some embodiments, the identity of one portion of a gene rearrangement is previously known (e.g., the portion of a gene rearrangement that is to be targeted by the gene-specific primers) and the sequence of the other portion may be determined using methods disclosed herein. In some embodiments, a gene rearrangement can involve an oncogene. In some embodiments, a gene rearrangement can comprise a fusion oncogene.

### Samples

In some embodiments, a target nucleic acid is present in or obtained from an appropriate sample (e.g., a food sample, environmental sample, biological sample e.g., blood sample, etc.). In some embodiments, the target nucleic acid is a biological sample obtained from a subject. In some embodiments a sample can be a diagnostic sample obtained from a subject. In some embodiments, a sample can further comprise proteins, cells, fluids, biological fluids, preservatives, and/or other substances. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample can be obtained from a subject in need of treatment for a disease associated with a genetic alteration, e.g., cancer or a hereditary disease. In some embodiments, a known target sequence is present in a disease-associated gene.

In some embodiments, a sample is obtained from a subject in need of treatment for cancer. In some embodiments, the sample comprises a population of tumor cells, e.g., at least one tumor cell. In some embodiments, the sample comprises a tumor biopsy, including but not limited to, untreated biopsy tissue or treated biopsy tissue (e.g., formalin-fixed and/or paraffin-embedded biopsy tissue).

In some embodiments, the sample is freshly collected. In some embodiments, the sample is stored prior to being used in methods and compositions described herein. In some embodiments, the sample is an untreated sample. As used herein, “untreated sample” refers to a biological sample that has not had any prior sample pre-treatment except for dilution and/or suspension in a solution. In some embodiments, a sample is obtained from a subject and preserved or processed prior to being utilized in methods and compositions described herein. By way of non-limiting example, a sample can be embedded in paraffin wax, refrigerated, or frozen. A frozen sample can be thawed before determining the presence of a nucleic acid according to methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g., anti-coagulant or nuclease inhibitor) and any combination thereof. In some embodiments, a sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in methods and compositions described herein. Suitable methods and processes for processing, preservation, or treatment of samples for nucleic acid analysis may be used in the method disclosed herein. In some embodiments, a sample can be a clarified fluid sample. In some embodiments, a sample can be clarified by low-speed centrifugation (e.g., 3,000×g or less) and collection of the supernatant comprising the clarified fluid sample.

In some embodiments, a nucleic acid present in a sample can be isolated, enriched, or purified prior to being utilized in methods and compositions described herein. Suitable methods of isolating, enriching, or purifying nucleic acids from a sample may be used. For example, kits for isolation of genomic DNA from various sample types are commercially available (e.g., Catalog Nos. 51104, 51304, 56504, and 56404; Qiagen; Germantown, Md.). In some embodiments, methods described herein relate to methods of enriching for target nucleic acids, e.g., prior to a sequencing of the target nucleic acids. In some embodiments, a sequence of one end of the target nucleic acid to be enriched is not known prior to sequencing. In some embodiments, methods described herein relate to methods of enriching specific nucleotide sequences prior to determining the nucleotide sequence using a next-generation sequencing technology. In some embodiments, methods of enriching specific nucleotide sequences do not comprise hybridization enrichment.

### Target Genes (ALK, ROS1, RET) and Therapeutic Applications

In some embodiments of methods described herein, a determination of the sequence contiguous to a known oligonucleotide target sequence can provide information relevant to treatment of disease. Thus, in some embodiments, methods disclosed herein can be used to aid in treating disease. In some embodiments, a sample can be from a subject in need of treatment for a disease associated with a genetic alteration. In some embodiments, a known target sequence is a sequence of a disease-associated gene, e.g., an oncogene. In some embodiments, a sequence contiguous to a known oligonucleotide target sequence and/or the known oligonucleotide target sequence can comprise a mutation or genetic abnormality which is disease-associated, e.g., a SNP, an insertion, a deletion, and/or a gene rearrangement. In some embodiments, a sequence contiguous to a known target sequence and/or a known target sequence present in a sample comprised sequence of a gene rearrangement product. In some embodiments, a gene rearrangement can be an oncogene, e.g., a fusion oncogene.

Certain treatments for cancer are particularly effective against tumors comprising certain oncogenes, e.g., a treatment agent which targets the action or expression of a given fusion oncogene can be effective against tumors comprising that fusion oncogene but not against tumors lacking the fusion oncogene. Methods described herein can facilitate a determination of specific sequences that reveal oncogene status (e.g., mutations, SNPs, and/or rearrangements). In some embodiments, methods described herein can further allow the determination of specific sequences when the sequence of a flanking region is known, e.g., methods described herein can determine the presence and identity of gene rearrangements involving known genes (e.g., oncogenes) in which the precise location and/or rearrangement partner are not known before methods described herein are performed.

In some embodiments, a subject is in need of treatment for lung cancer. In some embodiments, e.g., when the sample is obtained from a subject in need of treatment for lung cancer, the known target sequence can comprise a sequence from a gene selected from the group of ALK, ROS1, and RET. Accordingly, in some embodiments, gene rearrangements result in fusions involving the ALK, ROS1, or RET. Non-limiting examples of gene arrangements involving ALK, ROS1, or RET are described in, e.g., Soda et al. Nature 2007 448561-6: Rikova et al. Cell 2007131:1190-1203; Kohno et al. Nature Medicine 2012 18:375-7; Takouchi et al. Nature Medicine 201218:378-81; which are incorporated by reference herein in their entireties. However, it should be appreciated that the precise location of a gene rearrangement and the identity of the second gene involved in the rearrangement may not be known in advance. Accordingly, in methods described herein, the presence and identity of such rearrangements can be detected without having to know the location of the rearrangement or the identity of the second gene involved in the gene rearrangement.

In some embodiments, the known target sequence can comprise sequence from a gene selected from the group of: ALK, ROS1, and RET.

In some embodiments, the presence of a gene rearrangement of ALK in a sample obtained from a tumor in a subject can indicate that the tumor is susceptible to treatment with a treatment selected from the group consisting of: an ALK inhibitor; crizotinib (PF-02341066); AP26113; LDK378; 3-39; AF802; IPI-504; ASP3026; AP-26113; X-396; GSK-1838705A; CH5424802; diamino and aminopyrimidine inhibitors of ALK kinase activity such as NVP-TAE684 and PF-02341066 (see, e.g., Galkin et al., Proc Natl Acad Sci USA, 2007, 104:270-275; Zou et al., Cancer Res, 2007, 67:4408-4417; Hallberg and Palmer F1000 Med Reports 20113:21; Sakamoto et al., Cancer Cell 201119:679-690; and molecules disclosed in WO 04/079326). All of the foregoing references are incorporated by reference herein in their entireties. An ALK inhibitor can include any agent that reduces the expression and/or kinase activity of ALK or a portion thereof, including, e.g., oligonucleotides, small molecules, and/or peptides that reduce the expression and/or activity of ALK or a portion thereof. As used herein “anaplastic lymphoma kinase” or “ALK” refers to a transmembrane tyROS line kinase typically involved in neuronal regulation in the wildtype form. The nucleotide sequence of the ALK gene and mRNA are known for a number of species, including human (e.g., as annotated under NCBI Gene ID: 238).

In some embodiments, the presence of a gene rearrangement of ROS1 in a sample obtained from a tumor in a subject can indicate that the tumor is susceptible to treatment with a treatment selected from the group consisting of: a ROS1 inhibitor and an ALK inhibitor as described herein above (e.g., crizotinib). A ROS1 inhibitor can include any agent that reduces the expression and/or kinase activity of ROS1 or a portion thereof, including, e.g., oligonucleotides, small molecules, and/or peptides that reduce the expression and/or activity of ROS1 or a portion thereof. As used herein “c-ros oncogene 1” or “ROS1” (also referred to in the art as ros-1) refers to a transmembrane tyrosine kinase of the sevenless subfamily and which interacts with PTPN6. Nucleotide sequences of the ROS1 gene and mRNA are known for a number of species, including human (e.g., as annotated under NCBI Gene ID: 6098).

In some embodiments, the presence of a gene rearrangement of RET in a sample obtained from a tumor in a subject can indicate that the tumor is susceptible to treatment with a treatment selected from the group consisting of: a RET inhibitor; DP-2490, DP-3636, SU5416; BAY 43-9006, BAY 73-4506 (regorafenib), ZD6474, NVP-AST487, sorafenib, RPI-1, XL184, vandetanib, sunitinib, imatinib, pazopanib, axitinib, motesanib, gefitinib, and withaferin A (see, e.g., Samadi et al., Surgery 2010148:1228-36; Cuccuru et al., JNCI 2004 13:1006-1014; Akeno-Stuart et al., Cancer Research 200767:6956; Grazma et al., J Clin Oncol 201028:15s 5559; Mologni et al., J Mol Endocrinol 200637:199-212; Calmomagno et al., Journal NCI 200698:326-334; Mologni, Curr Med Chem 201118:162-175; and the compounds disclosed in WO 06/034833; US Patent Publication 2011/0201598 and U.S. Pat. No. 8,067,434). All of the foregoing references are incorporated by reference herein in their entireties. A RET inhibitor can include any agent that reduces the expression and/or kinase activity of RET or a portion thereof, including, e.g., oligonucleotides, small molecules, and/or peptides that reduce the expression and/or activity of RET or a portion thereof. As used herein, “rearranged during transfection” or “RET” refers to a receptor tyrosine kinase of the cadherin superfamily which is involved in neural crest development and recognizes glial cell line-derived neurotrophic factor family signaling molecules. Nucleotide sequences of the RET gene and mRNA are known for a number of species, including human (e.g., as annotated under NCBI Gene ID: 5979).

Further non-limiting examples of applications of methods described herein include detection of hematological malignancy markers and panels thereof (e.g., including those to detect genomic rearrangements in lymphomas and leukemias), detection of sarcoma-related genomic rearrangements and panels thereof; and detection of IGH/TCR gene rearrangements and panels thereof for lymphoma testing.

In some embodiments, methods described herein relate to treating a subject having or diagnosed as having, e.g., cancer with a treatment for cancer. Subjects having cancer can be identified by a physician using current methods of diagnosing cancer. For example, symptoms and/or complications of lung cancer which characterize these conditions and aid in diagnosis are well known in the art and include but are not limited to, weak breathing, swollen lymph nodes above the collarbone, abnormal sounds in the lungs, dullness when the chest is tapped, and chest pain. Tests that may aid in a diagnosis of, e.g., lung cancer include, but are not limited to, x-rays, blood tests for high levels of certain substances (e.g., calcium), CT scans, and tumor biopsy. A family history of lung cancer, or exposure to risk factors for lung cancer (e.g., smoking or exposure to smoke and/or air pollution) can also aid in determining if a subject is likely to have lung cancer or in making a diagnosis of lung cancer.

Cancer can include, but is not limited to, carcinoma, including adenocarcinoma, lymphoma, blastoma, melanoma, sarcoma, leukemia, squamous cell cancer, small-cell lung cancer, non-small cell lung cancer, gastrointestinal cancer, Hodgkin's and non-Hodgkin's lymphoma, pancreatic cancer, glioblastoma, basal cell carcinoma, biliary tract cancer, bladder cancer, brain cancer including glioblastomas and medulloblastomas; breast cancer, cervical cancer, choriocarcinoma; colon cancer, colorectal cancer, endometrial carcinoma, endometrial cancer; esophageal cancer, gastric cancer; various types of head and neck cancers, intraepithelial neoplasms including Bowen's disease and Paget's disease; hematological neoplasms including acute lymphocytic and myelogenous leukemia; Kaposi's sarcoma, hairy cell leukemia; chronic myelogenous leukemia, AIDS-associated leukemias and adult T-cell leukemia lymphoma; kidney cancer such as renal cell carcinoma, T-cell acute lymphoblastic leukemia/lymphoma, lymphomas including Hodgkin's disease and lymphocytic lymphomas; liver cancer such as hepatic carcinoma and hepatoma, Merkel cell carcinoma, melanoma, multiple myeloma; neuroblastomas; oral cancer including squamous cell carcinoma; ovarian cancer including those arising from epithelial cells, sarcomas including leiomyosarcoma, rhabdomyosarcoma, liposarcoma, fibROS1arcoma, and osteosarcoma; pancreatic cancer; skin cancer including melanoma, stromal cells, germ cells and mesenchymal cells; pROS1tate cancer, rectal cancer; vulval cancer, renal cancer including adenocarcinoma; testicular cancer including germinal tumors such as seminoma, non-seminoma (teratomas, choriocarcinomas), stromal tumors, and germ cell tumors; thyroid cancer including thyroid adenocarcinoma and medullar carcinoma; esophageal cancer, salivary gland carcinoma, and Wilms' tumors. In some embodiments, the cancer can be lung cancer.

### Multiplex Methods

Methods described herein can be employed in a multiplex format. In embodiments of methods described herein, multiplex applications can include determining the nucleotide sequence contiguous to one or more known target nucleotide sequences. As used herein, “multiplex amplification” refers to a process that involves simultaneous amplification of more than one target nucleic acid in one or more reaction vessels. In some embodiments, methods involve subsequent determination of the sequence of the multiplex amplification products using one or more sets of primers. Multiplex can refer to the detection of between about 2-1,000 different target sequences in a single reaction. As used herein, multiplex refers to the detection of any range between 2-1,000, e.g., between 5-500, 25-1,000, or 10-100 different target sequences in a single reaction, etc. The term “multiplex” as applied to PCR implies that there are primers specific for at least two different target sequences in the same PCR reaction.

In some embodiments, target nucleic acids in a sample, or separate portions of a sample, can be amplified with a plurality of primers (e.g., a plurality of first and second target-specific primers). In some embodiments, the plurality of primers (e.g., a plurality of first and second target-specific primers) can be present in a single reaction mixture, e.g., multiple amplification products can be produced in the same reaction mixture. In some embodiments, the plurality of primers (e.g., a plurality of sets of first and second target-specific primers) can specifically anneal to known target sequences comprised by separate genes. In some embodiments, at least two sets of primers (e.g., at least two sets of first and second target-specific primers) can specifically anneal to different portions of a known target sequence. In some embodiments, at least two sets of primers (e.g., at least two sets of first and second target-specific primers) can specifically anneal to different portions of a known target sequence comprised by a single gene. In some embodiments, at least two sets of primers (e.g., at least two sets of first and second target-specific primers) can specifically anneal to different exons of a gene comprising a known target sequence. In some embodiments, the plurality of primers (e.g., first target-specific primers) can comprise identical 5′ tag sequence portions.

In embodiments of methods described herein, multiplex applications can include determining the nucleotide sequence contiguous to one or more known target nucleotide sequences in multiple samples in one sequencing reaction or sequencing run. In some embodiments, multiple samples can be of different origins, e.g., from different tissues and/or different subjects. In such embodiments, primers (e.g., tailed random primers) can further comprise a barcode portion. In some embodiments, a primer (e.g., a tailed random primer) with a unique barcode portion can be added to each sample and ligated to the nucleic acids therein; the samples can subsequently be pooled. In such embodiments, each resulting sequencing read of an amplification product will comprise a barcode that identifies the sample containing the template nucleic acid from which the amplification product is derived.

### Molecular Barcodes

In some embodiments, primers may contain additional sequences such as an identifier sequence (e.g., a barcode, an index), sequencing primer hybridization sequences (e.g., Rd1), and adapter sequences. In some embodiments the adapter sequences are sequences used with a next generation sequencing system. In some embodiments, the adapter sequences are P5 and P7 sequences for Illumina-based sequencing technology. In some embodiments, the adapter sequence are P1 and A compatible with Ion Torrent sequencing technology.

In some embodiments, as used herein, “molecular barcode,” “molecular barcode tag,” and “index” may be used interchangeably, and generally refer to a nucleotide sequence of a nucleic acid that is useful as an identifier, such as, for example, a source identifier, location identifier, date or time identifier (e.g., date or time of sampling or processing), or other identifier of the nucleic acid. In some embodiments, such molecular barcode or index sequences are useful for identifying different aspects of a nucleic acid that is present in a population of nucleic acids. In some embodiments, molecular barcode or index sequences may provide a source or location identifier for a target nucleic acid. For example, a molecular barcode or index sequence may serve to identify a patient from whom a nucleic acid is obtained. In some embodiments, molecular barcode or index sequences enable sequencing of multiple different samples on a single reaction (e.g., performed in a single flow cell). In some embodiments, an index sequence can be used to orientate a sequence imager for purposes of detecting individual sequencing reactions. In some embodiments, a molecular barcode or index sequence may be 2 to 25 nucleotides in length, 2 to 15 nucleotides in length, 2 to 10 nucleotides in length, 2 to 6 nucleotides in length. In some embodiments, a barcode or index comprise at least 2, 3, 4, 5, 6, 7, 8, 9, 10, 11, 12, 13, 14, 15, 16, 17, 18, 19, 20, 21, 22, 23, 24, or at least 25 nucleotides.

In some embodiments, when a population of tailed random primers is used in accordance with methods described herein, multiple distinguishable amplification products can be present after amplification. In some embodiments, because tailed random primers hybridize at various positions throughout nucleic acid molecules of a sample, a set of target-specific primers can hybridize (and amplify) the extension products created by more than 1 hybridization event, e.g., one tailed random primer may hybridize at a first distance (e.g., 100 nucleotides) from a target-specific primer hybridization site, and another tailed random primer can hybridize at a second distance (e.g., 200 nucleotides) from a target-specific primer hybridization site, thereby resulting in two amplification products (e.g., a first amplification product comprising about 100 bp and a second amplification product comprising about 200 bp). In some embodiments, these multiple amplification products can each be sequenced using next generation sequencing technology. In some embodiments, sequencing of these multiple amplification products is advantageous because it provides multiple overlapping sequence reads that can be compared with one another to detect sequence errors introduced during amplification or sequencing processes. In some embodiments, individual amplification products can be aligned and where they differ in the sequence present at a particular base, an artifact or error of PCR and/or sequencing may be present.

### Computer and Control Equipment

The systems provided herein include several components, including sensors, environmental control systems (e.g., heaters, fans), robotics (e.g., an XY positioner), etc. which may operate together at the direction of a computer, processor, microcontroller or other controller. The components may include, for example, an XY positioner, a liquid handling devices, microfluidic pumps, linear actuators, valve drivers, a door operation system, an optics assembly, barcode scanners, imaging or detection system, touchscreen interface, etc.

In some cases, operations such as controlling operations of a systems and/or components provided therein or interfacing therewith may be implemented using hardware, software or a combination thereof. When implemented in software, the software code can be executed on any suitable processor or collection of processors, whether provided in a single component or distributed among multiple components. Such processors may be implemented as integrated circuits, with one or more processors in an integrated circuit component. A processor may be implemented using circuitry in any suitable format.

A computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable, mobile or fixed electronic device, including the system itself.

In some cases, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. In other examples, a computer may receive input information through speech recognition or in other audible format, through visible gestures, through haptic input (e.g., including vibrations, tactile and/or other forces), or any combination thereof.

One or more computers may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

The various methods or processes outlined herein may be coded as software that is executable on one or more processors that employ any one of a variety of operating systems or platforms. Such software may be written using any of a number of suitable programming languages and/or programming or scripting tools, and may be compiled as executable machine language code or intermediate code that is executed on a framework or virtual machine.

One or more algorithms for controlling methods or processes provided herein may be embodied as a readable storage medium (or multiple readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible storage medium) encoded with one or more programs that, when executed on one or more computers or other processors, perform methods that implement the various methods or processes described herein.

In some embodiments, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the methods or processes described herein. As used herein, the term “computer-readable storage medium” encompasses only a computer-readable medium that can be considered to be a manufacture (e.g., article of manufacture) or a machine. Alternatively or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computer or other processor to implement various aspects of the methods or processes described herein. Additionally, it should be appreciated that according to one aspect of this embodiment, one or more programs that when executed perform a method or process described herein need not reside on a single computer or processor, but may be distributed in a modular fashion amongst a number of different computers or processors to implement various procedures or operations.

Executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. Non-limiting examples of data storage include structured, unstructured, localized, distributed, short-term and/or long term storage. Non-limiting examples of protocols that can be used for communicating data include proprietary and/or industry standard protocols (e.g., HTTP, HTML, XML, JSON, SQL, web services, text, spreadsheets, etc., or any combination thereof). For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

In some embodiments, information related to the operation of the system (e.g., temperature, imaging or optical information, fluorescent signals, component positions (e.g., heated lid position, rotary valve position), liquid handling status, barcode status, bay access door position or any combination thereof) can be obtained from one or more sensors or readers associated with the system (e.g., located within the system), and can be stored in computer-readable media to provide information about conditions during a process (e.g., an automated library preparation process). In some embodiments, the readable media comprises a database. In some embodiments, said database contains data from a single system (e.g., from one or more bays). In some embodiments, said database contains data from a plurality of systems. In some embodiments, data is stored in a manner that makes it tamper-proof. In some embodiments, all data generated by the system is stored. In some embodiments, a subset of data is stored.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation —such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

What is claimed:

1. An apparatus for independently manipulating the temperature of a plurality of reaction vessels, the apparatus comprising:
a base assembly comprising i) a plurality of receptacles, each receptacle being configured to have disposed therein a reaction vessel, and ii) a plurality of thermoelectric devices, wherein each receptacle comprises a thermal jacket in thermal communication with at least one of the plurality of thermoelectric devices, wherein the thermal jacket has a first thermal transfer surface configured to surround at least a portion of a reaction vessel to facilitate thermal exchange between the reaction vessel and the jacket.

2. The apparatus of claim 1, wherein the thermal jacket has a second thermal transfer surface that interfaces with the at least one thermoelectric device to facilitate heat exchange between the thermal jacket and the at least one thermoelectric device.

3. The apparatus of claim 1, wherein the thermal jacket has a second thermal transfer surface in contact with a thermal pad that interfaces with the at least one thermoelectric device to facilitate heat exchange between the thermal jacket and the at least one thermoelectric device.

4. The apparatus of claim 3, wherein the thermal pad is composed of a ceramic-filled, high temperature silicone rubber coated on electrical fiberglass or a polyimide film or other film.

5. The apparatus of claim 4, wherein the thermal pad has a thermal resistance (Modified ASTM D5470) in a range of 3° C.-cm²/watt to 6° C.-cm²/watt.

6. The apparatus of claim 1, wherein the first thermal transfer surface contacts the reaction vessel.

7. The apparatus of claim 1, wherein the first thermal transfer surface is configured to surround a portion of the outer circumference of the reaction vessel.

8. The apparatus of claim 1, wherein the thermoelectric devices are Peltier devices configured to operate as a heat source or heat sink for the thermal jacket.

9. The apparatus of claim 1, wherein the thermoelectric devices are Peltier devices configured to cycle between heating and cooling operations relative to the thermal jacket.

10. The apparatus of claim 1, further comprising:
a cassette assembly comprising a plurality of reaction vessels

11. The apparatus of claim 10, wherein the cassette assembly is configured and arranged to interface with the base assembly such that each of the plurality of receptacles of the base assembly has disposed therein a corresponding reaction vessel of the cassette assembly

12. The apparatus of claim 1, further comprising:
a plurality of cassette assemblies, each cassette assembly comprising a plurality of reaction vessels

13. The apparatus of claim 12, wherein each cassette assembly is configured and arranged to interface with the base assembly such that each of the plurality of receptacles of the base assembly has disposed therein a corresponding reaction vessel of the cassette assembly

14. The apparatus of claim 12, wherein the plurality of cassette assemblies are disposed in a cartridge.

15. The apparatus of claim 14, wherein the cartridge comprises support structure comprising a channel system having a plurality of fluidic conduits, each of which fluidic conduit being in fluidic communication with a reaction vessel.

16. The apparatus of claim 14, wherein the cartridge comprises support structure comprising a channel system having a plurality of fluidic conduits, each of which fluidic conduit having a fluid outlet orifice that fluidically interfaces with an inlet port at the base of a reaction vessel.

17. The apparatus of claim 16, wherein the support structure has a plurality of openings aligned with each reaction vessel, each opening being configured to permit passage of a thermal jacket through the support structure to access and surround the reaction vessel.

18. The apparatus of claim 16, wherein the support structure comprises a plurality of elongate members, each elongate member extending from an outer edge of an opening to an inner position of the opening, wherein a fluidic conduit of the channel system extends through the elongate member from the outer edge to the inner position ending at a fluid outlet orifice at the inner position.

19. The apparatus of claim 18, wherein the thermal jacket has a keyway that aligns with the elongate member to permits passage of the jacket through the opening to access and surround the jacket.

20. The apparatus of claim 1, further comprising a thermal cover assembly.

21. The apparatus of claim 1, wherein the thermal cover assembly is configured to provide a heat source above each reaction vessel to prevent or minimize evaporation of a reaction solvent present in the reaction vessel.

22. The apparatus of claim 21, wherein the reaction solvent is water.

23. The apparatus of claim 20, wherein the thermal cover assembly has at least two independently thermally controlled zones.

24. An apparatus for managing temperature of a plurality of reaction vessels, the apparatus comprising:
a base assembly comprising a plurality of receptacles, wherein each receptacle is configured to have disposed therein a reaction vessel, and wherein each receptacle comprises a thermal jacket in thermal communication with a thermoelectric device, wherein the thermal jacket has a first thermal transfer surface configured to surround at least a portion of a reaction vessel to facilitate thermal exchange between the reaction vessel and the jacket; and
a thermal cover assembly configured to provide a heat source above each reaction vessel to prevent or minimize evaporation of a reaction solvent present in the reaction vessel, wherein the thermal cover assembly has at least two independently thermally controlled zones.

25. The apparatus of claim 24, wherein the thermal cover assembly has two independently thermally controlled zones.

26. The apparatus of claim 24, wherein each thermally controlled zone aligns with one or more reaction vessels of the plurality of reaction vessels.

27. The apparatus of claim 24, wherein the thermal cover assembly is configured with a plurality of openings to permit the passage of light, each opening being optically aligned above a corresponding reaction vessel.

28. The apparatus of claim 27, wherein the openings permit optical measurements from each reaction vessel.

29. The apparatus of claim 27, wherein the openings permit passage of light to or from the reaction vessel.

30. A method of manufacturing a circuit having a through-hole thermoelectric device (TED) mounted to a printed circuit board (PCB), wherein the through-hole TEC is packaged with a through-hole mount packaging and wherein the through-hole TED comprises a first side having a first surface area, the method comprising:
mounting the through-hole TED to the PCB in a mount area of the PCB arranged for mounting of the through-hole TED to the PCB, wherein the mount area comprises first holes corresponding to through-hole leads of the through-hole TED and a second hole having a size matching the first surface area of the first side of the through-hole TED, and wherein mounting the through-hole TED to the PCB comprises:
attaching the through-hole leads of the through-hole TED to the PCB via the first holes, and
arranging the through-hole TED on the PCB such that the first side of the through-hole TED is aligned with the second hole of the mounting area.

31. The method of claim 30, wherein the through-hole TED is a thermoelectric cooler (TEC) packaged with the through-hole mount packaging.

32. The method of claim 30, wherein the through-hole TED is arranged to create a temperature differential between the first side of the through-hole TED and a second side of the through-hole TED based on a power applied to the through-hole TED.

33. The method of claim 30, wherein the through-hole TED is arranged to create a thermal gradient in the through-hole TED via a Peltier effect.

34. The method of claim 30, wherein the through-hole TED is operable to adjust a temperature of the first side of the through-hole TED in response to application of power to the through-hole leads.

35. The method of claim 30, wherein arranging the through-hole TED on the PCB such that the first side is aligned with the second hole comprises arranging the through-hole TED such that the first side is disposed in the second hole.

36. The method of claim 30, herein arranging the through-hole TED on the PCB such that the first side is aligned with the second hole comprises arranging the through-hole TED such that a plane of the first side of the through-hole TED is substantially parallel to a plane of the second hole.

37. The method of claim 30, wherein:
the PCB comprises a plurality of the mount area, each mount area of the plurality comprising the first holes and the second hole; and
the method further comprises repeating the mounting for a plurality of through-hole TEDs to mount the plurality of through-hole TEDs in the plurality of mount areas.

38. The method of claim 30, wherein the mounting is performed by circuit manufacture equipment configured to perform the mounting.

39. The method of claim 30, wherein:
the first holes each comprise an electrically conductive material individually electrically connecting the first holes to conductive traces of the PCB; and
attaching the through-hole leads of the through-hole TED to the PCB via the first holes comprises soldering a lead of the through-hole leads to the electrically conductive material of a hole of the first holes.

40. An apparatus comprising:
a printed circuit board (PCB) comprising a mount area arranged to have mounted thereon a through-hole thermoelectric device (TED), the through-hole TED being packaged with a through-hole mount packaging, wherein the mount area comprises:
first holes corresponding to through-hole leads of the through-hole TED; and
a second hole having a size matching a first surface area of a first side of the through-hole TED.

41. The apparatus of claim 40, further comprising:
the through-hole TED,
wherein the through-hole TED is a thermoelectric cooler (TEC) packaged with the through-hole mount packaging.

42. The apparatus of claim 40, further comprising:
the through-hole TED,
wherein the through-hole TED is arranged to create a temperature differential between the first side of the through-hole TED and a second side of the through-hole TED based on a power applied to the through-hole TED.

43. The apparatus of claim 40, further comprising:
the through-hole TED,
wherein the through-hole TED is arranged to create a thermal gradient in the through-hole TED via a Peltier effect.

44. The apparatus of claim 40, further comprising:
the through-hole TED,
wherein the through-hole TED is operable to adjust a temperature of the first side of the through-hole TED in response to application of power to the through-hole leads.

45. The apparatus of claim 40, further comprising:
the through-hole TED mounted in the mount area of the PCB, the through-hole TED being mounted to the PCB with through-hole pins of the through-hole TED attached to the first holes of the PCB and the first side of the through-hole TED being aligned with the second hole of the PCB.

46. The apparatus of claim 45, further comprising:
the through-hole TED mounted in the mount area of the PCB, wherein the through-hole TED is mounted in the mount area such that the first side is disposed in the second hole.

47. The apparatus of claim 45, further comprising:
the through-hole TED mounted in the mount area of the PCB, wherein the through-hole TED is mounted to the PCB such that a plane of the first side of the through-hole TED is substantially parallel to a plane of a second hole.

48. The apparatus of claim 40, wherein the PCB comprises a plurality of the mount area, each mount area of the plurality comprising the first holes and the second hole.

49. The apparatus of claim 48, further comprising:
a plurality of the through-hole TED each mounted in a mount area of the plurality of mount areas, wherein each through-hole TED is mounted to a corresponding mount area of the plurality of mount areas with through-hole pins of the through-hole TED attached to the first holes of the corresponding mount area and the first side of the through-hole TED being positioned to correspond to the second hole of the corresponding mount area.

50. The apparatus of claim 40, wherein:
the first holes of the mount area comprise one hole and another hole, the one hole and the other hole each comprising a conductive material;
the through-hole TED comprises a second side opposite the first side; and
the through-hole TED is operable to heat the first side and cool the second side, or cool the first side and cool the second side, dependent on a direction of current applied to the through-hole leads of the through-hole TED; and
the apparatus further comprises:
a first conductive trace connected to the conductive material of the one hole and a second conductive trace connected to the conductive material of the other hole; and
at least one circuit to drive current to the one hole and the other hole in a direction to operate the through-hole TED to heat the first side and cool the second side or to cool the first side and cool the second side.

51. The apparatus of claim 50, further comprising:
the through-hole TED, wherein one lead of the through-hole leads is attached to the one hole of the mount area and another lead of the through-hole leads is attached to the other hole of the mount area.

52. An apparatus comprising:
a printed circuit board (PCB); and
a through-hole TED,
wherein the PCB comprises a mount area arranged to have mounted thereon a through-hole thermoelectric device (TED), the through-hole TED being packaged with a through-hole mount packaging, wherein the mount area comprises:
first holes corresponding to through-hole leads of the through-hole TED; and
a second hole having a size matching a first surface area of a first side of the through-hole TED; and
wherein the through-hole TED is mounted in the mount area of the PCB, the through-hole TED being mounted to the PCB with through-hole pins of the through-hole TED attached to the first holes of the PCB and the first side of the through-hole TED aligned with the second hole of the PCB.

53. A method of manufacturing a printed circuit board (PCB), the method comprising:
forming the PCB with a mount area arranged to have mounted thereon a through-hole thermoelectric device (TED), the through-hole TED being packaged with a through-hole mount packaging, wherein forming the PCB with the mount area comprises:
forming the PCB with first holes corresponding to through-hole leads of the through-hole TED; and
forming the PCB with a second hole having a size matching a first surface area of a first side of the through-hole TED.

54. The method of claim 53, wherein forming the PCB with the first holes and/or the second hole comprises excising material from the PCB to form the first holes and/or the second hole.

55. The method of claim 53, wherein forming the PCB with the first holes and/or the second hole comprises molding the PCB with the first holes and/or the second hole.

56. The method of claim 53, wherein forming the PCB with the mount area comprises forming the PCB with a plurality of the mount area, each mount area of the plurality comprising the first holes and the second hole.

57. The method of claim 53, wherein the forming is performed by manufacture equipment configured to perform the forming.

58. The method of claim 53, further comprising:
the through-hole TED,
wherein the through-hole TED is a thermoelectric cooler (TEC) packaged with the through-hole mount packaging.

59. The method of claim 53, further comprising:
the through-hole TED,
wherein the through-hole TED is arranged to create a temperature differential between the first side of the through-hole TED and a second side of the through-hole TED based on a power applied to the through-hole TED.

60. The method of claim 53, further comprising:
the through-hole TED,
wherein the through-hole TED is arranged to create a thermal gradient in the through-hole TED via a Peltier effect.

61. The method of claim 53, further comprising:
the through-hole TED,
wherein the through-hole TED is operable to adjust a temperature of the first side of the through-hole TED in response to application of power to the through-hole leads.
